SEQUENCE LISTING (77 sequences)

SEQ ID NO: 1 - RNA, 21 nt, Artificial Sequence
  Synthetic
agaauuugacaaggcacgucg

SEQ ID NO: 2 - RNA, 21 nt, Artificial Sequence
  Synthetic
aaacacgguacuguugaagga

SEQ ID NO: 3 - RNA, 21 nt, Artificial Sequence
  Synthetic
auuuccuugguugcccagagc

SEQ ID NO: 4 - RNA, 21 nt, Artificial Sequence
  Synthetic
auagucugcaugggugacuga

SEQ ID NO: 5 - DNA, 3183 bp, Homo sapiens
actcgctccctcccccggatcccgactgggaagggggcggaggggacgacgtccgcgagaatgcaaaccacacaaaaccccgaagtggccgtccccgccgggctcgaacctctgcgaacacccgctgggctgctgctcccccttctttctgtctttcctttattttttggaagtgacaagggcccatctgcgcccagacccccgttcgccctctggcccgcgggcgaagaggggagagggagctgcccgcgcagtccccgggctcgggccattcgaccgggtggggggtgacggggggtcgcggaggcggccaggctaggaaaggggagaagagccgaagacagcacagacttgagcggcggcgccgcgggaggcactagagcgggcccgagcgaaacataaacaaacgcacgcacacccgagctcgggctctgaccgcggctcggctgcgccagctccggccgctgcccgctttaaaggcgcggccgcccctcccccgggcgcccctcccccttctccccgccccgccgcctagcccgggagggacctgcggctgggcggcggggggagggggctgccccgcgcgaggccgtcgattcgctcgcggctccatcgcggcctggccggggggcggtgtctgctgcgccaggttcgctggccgcacgtcttcaggtcctcctgttcctgggaggcgggcgcggcaggactgggaggtggcggcagcgggcgaggactcgccgaggacggggctccggcccgggataaccaactctccttctctcttctttggtgcttccccaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgctgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttcc

SEQ ID NO: 6 - DNA, 2889 bp, Mus musculus
gctccctgtgagtggctataactttgtgctgctgccgcggccgccctgctcgtggaagggaggaggaggaatgtggaaggcggcggcgcagcacaggctgacaggcggttcctccggcgggctgcggcggcggcccgagagtcccctcgtcgcggtgccctgggctcgcgcggaatcgtacgccctcccgcctcgttcctgaagggaaggagccgagctggagctcgaaccttcgcggtgccccgttcctcccccgccgcacccagcctgggctcgagaggagagagcaagagcccaagccgcgggcggcgggcaggcggcgaagatggcagaggcaccagcctccccggtcccgctctctccgctcgaagtggagctggacccagagttcgagccacagagtcggccacgctcctgtacgtggcccctgcagaggccggagctgcaggcgagcccggccaagccctcgggggagacggccgcagactccatgatccccgaggaggacgacgatgaagacgacgaggacggcggcggccgagccagctcggccatggtgatcggtggcggcgtgagcagcacgctgggttccgggctgctcctcgaggattcggccatgctgctggctccaggagggcaggacctcgggtcggggccagcgtccgccgcaggcgctctgagtgggggcacgccgacgcagctgcagcctcagcagccactgccacagccgcagccgggggcggctgggggctctgggcaaccaaggaaatgctcctcgcggcggaatgcctgggggaacctgtcctatgccgacctgatcacccgcgccatcgagagctccccggacaaacggctcactttgtcccagatctacgagtggatggtgcgctgtgtgccctacttcaaggataagggcgacagcaacagctctgcgggctggaagaactccatccggcacaacctgtccctgcacagccgcttcatgcgcgttcagaatgaaggcacgggcaagagctcttggtggatcatcaaccccgatgggggaaagagcgggaaggccccccggcggcgtgcggtctccatggacaacagcaacaagtacaccaagagccgaggccgggcagccaagaagaaggcggccctgcaggctgccccagagtcggcagacgacagtccttcccagctctccaagtggcctggcagccccacgtcccgcagcagcgacgagctggatgcgtggaccgacttccgctcgcgcaccaattccaacgccagcaccgtgagcggccgcctgtcgcccatcctggcaagcacggagctggatgacgtccaggatgatgatggacccctgtcccccatgctgtacagcagctctgccagcctgtcgccctccgtgagcaagccgtgtactgtggagcttccgcggctgacggacatggccggcaccatgaatctgaatgatgggctggccgagaacctcatggacgacctgctggataacatcgcgctcccgccatcgcagccatcgcctcctggcgggcttatgcagcggggctccagcttcccatataccgccaagagctccggcctgggctccccaaccggctccttcaacagtaccgtgtttggaccttcgtctctgaactccttgcgtcagtcacccatgcagactatccaggagaacagaccagccaccttctcttccgtgtcacactacggcaaccagacactccaagacctgcttgcttcagactcactcagccacagcgacgtcatgatgacccagtcggaccccttgatgtctcaggctagcaccgccgtgtccgcccagaatgcccgccggaacgtgatgcttcgcaacgatccaatgatgtcctttgctgcccagcctacccaggggagtttggtcaatcagaacttgctccaccaccagcaccaaacccagggcgctcttggtggcagccgtgccttgtcaaattctgtcagcaacatgggcttgagtgactccagcagccttggctcagccaaacaccagcagcagtctcccgccagccagtctatgcaaaccctctcggactctctctcaggctcctcactgtattcagctagtgcaaaccttcccgtcatgggccacgataagttccccagtgacttggacctggacatgttcaatgggagcttggaatgtgacatggagtccatcatccgtagtgaactcatggatgctgacgggttggattttaactttgactccctcatctccacacagaacgttgttggtttgaatgtggggaacttcactggtgctaagcaggcctcatctcaaagctgggtaccaggctgaaggatcactgaggaaaggggaaatgggcaaagcagaccctcaaactgacggaagacctacagagaaaaccctttgccaaatctgctctcagcaagtggacagtgatccgtttacagcttgacacctttgagactcccacgccgcttccctaacccagcagagactgttagcagccctggccctgggtgaagcccttacccgtggaacagaactctataacatatgcaaaatctaactcatctgcaagtgacctgccagcgtggacagcacccatcagcaccccccactccttcagagcacaccgggaccccgttggcgactccgtcgtgttttactatcatgatggtttaggggatattttaagtgtcgtcttgtgtttgtttcctttgaccctctgagttttctacacacagtaacctgcagtatttttctgttgaaaatgttaactgtcctttccctagcacacttaaaagcagaaggaaggtgttatatcagttcccagtctggccttgagcatcgcaagcttttgagcctgtgggacccca

SEQ ID NO: 7 - DNA, 7341 bp, Homo sapiens
gcgcgaggccgtcgattcgctcgcggctccatcgcggcctggccggggggcggtgtctgctgcgccaggttcgctggccgcacgtcttcaggtcctcctgttcctgggaggcgggcgcggcaggactgggaggtggcggcagcgggcgaggactcgccgaggacggggctccggcccgggataaccaactctccttctctcttctttggtgcttccccaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 8 - DNA, 7314 bp, Homo sapiens
ctgctcgtggaagggaggaggaggaatgtggaaggtggcggcgcgcgagctgacaggcggttcctcgggcgggctgcggcgacggcccgagcgcttctccttcgccgaggttgcgcgccccctcctcgccgtgccccgggcccgcgcgggatcgtgcgtcgtcccgccgcgtccctgaagggaaggaagcgaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 9 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 10 - DNA, 2889 bp, Mus musculus; identical to SEQ ID NO: 6

SEQ ID NO: 11 - protein, 672 aa, Mus musculus
MAEAPASPVPLSPLEVELDPEFEPQSRPRSCTWPLQRPELQASPAKPSGETAADSMIPEEDDDEDDEDGGGRASSAMVIGGGVSSTLGSGLLLEDSAMLLAPGGQDLGSGPASAAGALSGGTPTQLQPQQPLPQPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQAAPESADDSPSQLSKWPGSPTSRSSDELDAWTDFRSRTNSNASTVSGRLSPILASTELDDVQDDDGPLSPMLYSSSASLSPSVSKPCTVELPRLTDMAGTMNLNDGLAENLMDDLLDNIALPPSQPSPPGGLMQRGSSFPYTAKSSGLGSPTGSFNSTVFGPSSLNSLRQSPMQTIQENRPATFSSVSHYGNQTLQDLLASDSLSHSDVMMTQSDPLMSQASTAVSAQNARRNVMLRNDPMMSFAAQPTQGSLVNQNLLHHQHQTQGALGGSRALSNSVSNMGLSDSSSLGSAKHQQQSPASQSMQTLSDSLSGSSLYSASANLPVMGHDKFPSDLDLDMFNGSLECDMESIIRSELMDADGLDFNFDSLISTQNVVGLNVGNFTGAKQASSQSWVPG

SEQ ID NO: 12 - DNA, 21 bp, Mus musculus
agccgtgccttgtcaaattct

SEQ ID NO: 13 - DNA, 21 bp, Mus musculus
tccttcaacagtaccgtgttt

SEQ ID NO: 14 - DNA, 21 bp, Mus musculus
gctctgggcaaccaaggaaat

SEQ ID NO: 15 - DNA, 21 bp, Mus musculus
tcagtcacccatgcagactat

SEQ ID NO: 16 - DNA, 1099 bp, Homo sapiens
agccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctccatattgcaacagtcctcactcccacaccaggtccccgctccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccacctcctgagcccctcaatcctatcacagtctactgacttttcccattcagctgtgagtgcccaaccctatcccagagaccttgatgcttggcctcccaatcttgccctaggatacccagatgccaaccagacacctccttcttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgattccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctccca

SEQ ID NO: 17 - DNA, 2653 bp, Homo sapiens
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 18 - DNA, 1038 bp, Homo sapiens
atttgaggccatataaagtcacctgaggccctctccaccacagcccaccagtgaccacgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctaggctctggggggccccgctgcagcccacactgggtgtggtgccccaggcctctgtgccactcctcacacacccggcccagtgggagcctgtcctggttcctgaggcacatcctaacgcaagtctgaccatgtatgtctgcgcccctgtcccccaccctgaccctcccatggccctctccaggactcccacccggcagatcggctctattgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccggtaaaggctgagatgaagtggactgagtagaactggaggacaggagtcgacgtgagttcctgggagtctccagagatggggcctggaggcctggaggaaggggccaggcctcacattcgtggggctccctgaatggcagcctcagcacagcgtaggcccttaataaacacctgttggataagccagaaaaaaaaaaaaaaaa

SEQ ID NO: 19 - DNA, 3163 bp, Homo sapiens
tcaatcccttaattaaatagcttcccctctacaggcttttgaagtggtagcagttcctcctaactcctgccagaaacagctctcctcaacatgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaactcatcatgtattctgcgcatgacactactgtgagtggcctacagatggcgctagatgtttacaacggactccttcctccctatgcttcttgccacttgacggaattgtactttgagaagggggagtactttgtggagatgtactatcggaatgagacgcagcacgagccgtatcccctcatgctacctggctgcagccccagctgtcctctggagaggtttgctgagctggttggccctgtgatccctcaagactggtccacggagtgtatgaccacaaacagccatcaaggtactgaagacagtacagattagtgtgcacagagatctctgtagaaggagtagctgccctttctcagggcagatgatgctttgagaacatactttggccattacccccagctttgaggaaaatgggctttggatgattattttatgttttagggacccccaacctcaggcaattcctacctcttcacctgaccctgcccccacttgccataaaacttagctaagttttgttttgtttttcagcgttaatgtaaaggggcagcagtgccaaaatataatcagagataaagcttaggtcaaagttcatagagttcccatgaactatatgactggccacacaggatcttttgtatttaaggattctgagattttgcttgagcaggattagataaggctgttctttaaatgtctgaaatggaacagatttcaaaaaaaaaccccacaatctaggatgggaacaaggaaggaaagatgtgaataggctgatgggcaaaaaaccaatttacccatcagttccagccttctctcaaggagaggcaaagaaaggagatacagtggagacatctggaaagttttctccactggaaaactgctactatctgtttttatatttctgttaaaatatatgaggctacagaactaaaaattaaaacctctttgtgtcccttggtcctggaacatttatgttccttttaaagaaacaaaaatcaaactttacagaaagatttgatgtatgtaatacatatagcagctcttgaagtatatatatcatagcaaataagtcatctgatgagaacaagctatttgggcacaacacatcaggaaagagagcaccacgtgatggagtttctctagaagctccagtgataagagatgttgactctaaagttgatttaaggccaggcatggtggtttacgcctataatcccagcattttgggagtccgaggtgggcagatcacttgagctcaggaggtcaagatcagcctgggcaacatggtgaaacctggtctctacataaaatacaaaaacttagatgggcatggtggtgtgtgcctatagtcccactacttgtggggctaaggcaggaggatcacttgagccccggaggtcgaggctacagtgagccaagagtgcactactgtactccagccagggcaagagagcgagaccctgtctcaataaataaataaataaataaataaataaataaataaataaataaaaacaaagttgattaagaaaggaagtataggccaggcacagtggctcacacctgtaatccttgcattttggaaggctgaggcaggaggatcactttaggcctggtgtgttcaagaccagcctggtcaacatagtgagacactgtctctaccaaaaaaaggaaggaagggacacatatcaaactgaaacaaaattagaaatgtaattatgttctaagtgcctccaagttcaaaacttattggaatgttgagagtgtggttacgaaatacgttaggaggacaaaaggaatgtgtaagtctttaatgccgatatcttcagaaaacctaagcaaacttacaggtcctgctgaaactgcccactctgcaagaagaaatcatgatatagctttgccatgtggcagatctacatgtctagagaacactgtgctctattaccattatggataaagatgagatggtttctagagatggtttctactggctgccagaatctagagcaaagccatccccgctcctggttggtcacagaatgactgacaaagacatcgattgatatgcttctttgtgttatttccctcccaagtaaatgtttgtccttgggtccattttctatgcttgtaactgtcttctagcagtgagccaaatgtaaaatagtgaataaagtcattattaggaagttcaaaagcattgcttttataatgaacttagaaaaac

SEQ ID NO: 20 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 21 - DNA, 2655 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 22 - DNA, 1209 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 23 - protein, 104 aa, Homo sapiens
MWVPVVFLTLSVTWIGERGHGWGDAGEGASPDCQAEALSPPTQHPSPDRELGSFLSLPAPLQAHTPSPSILQQSSLPHQVPAPSHLPQNFLPIAQPAPCSQLLY

SEQ ID NO: 24 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 25 - protein, 114 aa, Homo sapiens
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 26 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 27 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 28 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 29 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 30 - DNA, 2300 bp, Homo sapiens
ggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggtaccctggctaaagccgggccggagccctctgccctctcatgcccgcagccagcctgggctgtgcaacatgtacaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggtaaaaaaaaaaaaaaaaaaaaaa

SEQ ID NO: 31 - DNA, 2777 bp, Homo sapiens
gaattcgggaaaagaccgaagaaggaggctggagagaccaggatccttccagctgaacaaagtcagccacaaagcagactagccagccggctacaattggagtcagagtcccaaagacatgggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtacttgtttgggaaaatggctaggacatcccgacaagtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgacgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctgatcccccgtagaaatccccctttctctaatagcgaggctcctctaaccacacagcctacaatgctgcgtctcccatcttaactctttgcctttgccaccaactggccctcttcttacttgatgagtgtaacaagaaaggagagtcttgcagtgattaaggtctctctttggactctcccctcttatgtacctcttttagtcattttgcttcatagctggttcctgctagaaatgggaaatgcctaagaagatgacttcccaactcgaagtcacaaaggaatggaggctctaattgaattttcaagcatctcctgaggatcagaaagtaatttcttctcaaagggtacttccactgatggaaacaaagtggaaggaaagaaggtcaggtacagagaaggaatgtctttggtcctcttgccatctataggggccaaatatattctctttggtgtacaaaatggaattcattctggtctctctattaccactgaagatagaagaaaaaagaatgtcagaaaaacaataagagcgtttgcccaaatctgcctattgcagctgggagaagggggtcaaagcaaggatctttcacccacagaaagagagcactgaccccgatggcgatggactactgaagccctaactcagccaaccttacttacagcataagggagcgtagaatctgtgtagacgaagggggcatctggccttacacctcgttagggaagagaaacagggtgttgtcagcatcttctcactcccttctccttgataacagctaccatgacaaccctgtggtttccaaggagctgagaatagaaggaaactagcttacatgagaacagactggcctgaggagcagcagttcctggtggctaatggtgtaacctgagatggccctctggtagacacaggatagataactctttggatagcatgtctttttttctgttaattagttgtgtactctggcctctgtcatatcttcacaatggtgctcatttcatggggtattatccattcagtcatcgtaggtgatttgaaggtcttgatttgttttagaatgatgcacatttcatgtattccagtttgtttattacttatttggggttgcatcagaaatgtctggagaataattctttgattatgactgttttttaaactaggaaaattggacattaagcatcacaaatgatattaaaaattggctagttgaatctattgggattttctacaagtattctgcctttgcagaaacagatttggtgaatttgaatctcaatttgagtaatctgatcgttctttctagctaatggaaaatgattttacttagcaatgttatcttggtgtgttaagagttaggtttaacataaaggttattttctcctgatatagatcacataacagaatgcaccagtcatcagctattcagttggtaagcttccaggaaaaaggacaggcagaaagagtttgagacctgaatagctcccagatttcagtcttttcctgtttttgttaactttgggttaaaaaaaaaaaaagtctgattggttttaattgaaggaaagatttgtactacagttcttttgttgtaaagagttgtgttgttcttttcccccaaagtggtttcagcaatatttaaggagatgtaagagctttacaaaaagacacttgatacttgttttcaaaccagtatacaagataagcttccaggctgcatagaaggaggagagggaaaatgttttgtaagaaaccaatcaagataaaggacagtgaagtaatccgtaccttgtgttttgttttgatttaataacataacaaataaccaa

SEQ ID NO: 32 - DNA, 1920 bp, Homo sapiens
gtcatcccctcagtctgtagcccttttgtgtgagtgcctggcaagggtgacgtggggctgtttctgcgggcacagctgcagcaattaccggagtggaggcagggcccaggcagcactgccctccaagatcttcccttgggcttttcagcagtaaggggacatgcaccccaagggcctccacttggcctgaccttgctgcgggggctctctgtccccaggaacagtagagatggcaagcttatcaagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggagtgctggttctcctcgcggtgctgcctgtgctcctcctgcagatcactgttggcctcatcttcctctgcctgcagtacagactgagaggaaaacttcgagcagagatagagaatctccaccggacttttgatccccactttctgagggtgccctgctggaagataaccctgtttgtaattgtgccggttcttggacccttggttgccttgatcatctgctacaactggctacatcgaagactagcagggcaattccttgaagagctactcttccacctggaagccctctctggctaaggacaggcaggtgcccctctctccatcagaggacacctgtactggagagcaacacaggatggtctctgccatgaactggaggccaggaatctcctcactgaaaattacagtatggtaactttgcaaatggtggttgtttcttccaagactccagccctgattgcgcaaaactgaaaggcatgtgaagggaaggaagaggaagagtgcaaaacattgaagagagagctgagtgagctgaagagtgaggatatgagtagccccaacccaaacctggagatggggagaaacctacagaatactagccagagctcctccttgtcttggcagcctactagggacctggggaagcaaaaacgaaagctgggcaacatgcctgctttagaatgttttccttctacttacacatcttccacaggtctcagaatctttccttcctctcatccttttctcctatcttcatatctatcagagtatccactgtttattcaacaactactacttgatggtcagacacaaacaaacaagctaggtgctaattaataaagatacgagttttggccgggtgcggtggctcacgcctgtaatcccagcactttgggaggccgaggcgggcgaatcacgaggtcaggagttcaagaccagcctggccaacatggtgaaaccccatctctactaaaaatacaaacaattaactgagcatagtggtgggcacctataataccagctactccggaggctgaggcaggagaatcgcttgaacccaggaggcagaggttgcagtgagctgagatcgtgccactgcactctagccggagtgacagagtaagactctgtctcaaaaataaataaataaataaataaataaataaataaataaataaataaaaaataataatacaagttttcataagcacacttctaaaaaaaaaaaa

SEQ ID NO: 33 - DNA, 469 bp, Homo sapiens
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 34 - DNA, 3488 bp, Homo sapiens
gcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgagcctgtttctgcgccggaccagtcgaggactctggacagtagaggccccgggacgaccgagctgatggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaatcatccttcgcagaacatgagtttatgggaatgccttttccctctggcactccagaacaaacctctatatgttgctgaaacacacaggccatttcattgagggaaaacataatatcttgaagaatattgttaaaaccttacttaaagcttgtttgttctagttagcaggaaatagtgttctttttaaaaagttgcacattaggaacagagtatatatgtacagttatacatacctctctctatatatacatgtatagtgagtgtggcttagtaatagatcacggcatgtttcccgctccaagagaattcactttaccttcagcagttaccgaggagctaaacatgctgccaaccagcttgtccaacaactccaggaaaactgtttttcaaaacgccatgtcctaggggccaagggaaatgctgttggtgagaatcgacctcactgtcagcgtttctccacctgaagtgatgatggatgagaaaaaacaccaccaaatgacaagtcacaccctccccattagtatcctgttaggggaaaatagtagcagagtcattgttacaggtgtactatggctgtatttttagagattaatttgtgtagattgtgtaaattcctgttgtctgaccttggtggtgggagggggagactatgtgtcatgatttcaatgattgtttaattgtaggtcaatgaaatatttgcttatttatattcagagatgtaccatgttaaagaggcgtcttgtattttcttcccatttgtaatgtatcttatttatatatgaagtaagttctgaaaactgtttatggtattttcgtgcatttgtgagccaaagagaaaagattaaaattagtgagatttgtatttatattagagtgcccttaaaataatgatttaagcattttactgtctgtaagagaattctaagattgtacataaagtcatatatatggaaatcctgttacttaaatagcatctgctcttctcttacgctctctgtctggctgtacgtctggtgttctcaatgcttttctagcaactgttggataataactagatctcctgtaattttgtagtagttgatgaccaatctctgtgactcgcttagctgaaacctaaggcaacatttccgaagaccttctgaagatctcagataaagtgaccaggctcacaactgtttttgaagaagggaaattcacactgtgcgttttagagtatgcaagaagaatataaataaataaaaatattctccatggagaatttgaacaaaaaaaaaaaaaaaa

SEQ ID NO: 35 - DNA, 5373 bp, Homo sapiens
agcagtttttgtaggtgaaaacaatgaagccaggtaatattgcaaggaggctgtaattttagcagacctaccaacaacactgatgtaggaagctcattattttaatttctggagccttttaattttttctttagaaagtgtataaataattgcagtgctgctttgcttccaaaactgggcagtgagttcaacaacaacgacaacaacagccgcagctcatcctggccgtcatggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactagctcagtcacaccgtcagtttcccaaatttgacaggccaccttcaaacatgctgctatgcagtttctgcatcatagaaaataaggaaccaaaggaagaaattcatgtcatggtgcaatgcacattttatctatttatttagttccattcaccatgaaggaagaggcactgagatccatcaatcaattggattatatactgatcagtagctgtgttcaattgcaggaatgtgtatatagattattcctgagtggagccgaagtaacagctgtttgtaactatcggcaataccaaattcatctcccttccaataatgcatcttgagaacacataggtaaatttgaactcaggaaagtcttactagaaatcagtggaagggacaaatagtcacaaaattttaccaaaacattagaaacaaaaaataaggagagccaagtcaggaataaaagtgactctgtatgctaacgccacattagaacttggttctctcaccaagctgtaatgtgatttttttttctactctgaattggaaatatgtatgaatatacagagaagtgcttacaactaatttttatttacttgtcacattttggcaataaatccctcttatttctaaattctaacttgtttatttcaaaactttatataatcactgttcaaaaggaaatattttcacctaccagagtgcttaaacactggcaccagccaaagaatgtggttgtagagacccagaagtcttcaagaacagccgacaaaaacattcgagttgaccccaccaagttgttgccacagataatttagatatttacctgcaagaaggaataaagcagatgcaaccaattcattcagtccacgagcatgatgtgagcactgctttgtgctagacattgggcttagcattgaaactataaagaggaatcagacgcagcaagtgcttctgtgttctggtagcaactcaacactatctgtggagagtaaactgaagatgtgcaggccaacattctggaaatcctatgtcaatgggtttggtttggaacctggacttctgcatttttaaaagttacccagagatgcttctaaagatgagccatagtctagaagattgtcaaccacaggagttcattgagtgggacagctagacacatacattggcagctacaatagtatcatgaattgcaatgatgtagtggggtataaaaggaaagcgatggatattgccggatgggcatggccagtgatgtttcacgtcattgaggtgacagctctgctggactttgaattacatatggaggctctccaggaagacgaagaagagaaggacattctaggcaaaaagaagactaggcacaaggcacacttatgtttgtctgttagcttttagttgaaaaagcaaaatacatgatgcaaagaaacctctccacgctgtgatttttaaaactacatactttttgcaactttatggttatgagtattgtagagaacaggagataggtcttagatgatttttatgttgttgtcagactctagcaaggtactagaaacctagcaggcattaataattgttgaggcaatgactctgaggctatatctgggccttgtcattatttatcatttatatttgtatttttttctgaaatttgagggccaagaaaacattgactttgactgaggaggtcacatctgtgccatctctgcaaatcaatcagcaccactgaaataactacttagcattctgctgagctttccctgctcagtagagacaaatatactcatcccccacctcagtgagcttgtttaggcaaccaggattagagctgctcaggttcccaacgtctcctgccacatcgggttctcaaaatggaaagaatggtttatgccaaatcacttttcctgtctgaaggaccactgaatggttttgtttttccatattttgcataggacgccctaaagactaggtgacttggcaaacacacaagtgttagtataattctttgcttctgcttctttttgaaaatcatgtttagatttgattttaagtcagaaattcactgaatgtcaggtaatcattatggagggagatttgtgtgtcaaccaaagtaattgtcccatggccccagggtatttctgttgtttccctgaaattctgcttttttagtcagctagattgaaaactctgaacagtagatgtttatatggcaaaatgcaagacaatctacaagggagattttaaggattttgagatgaaaaaacagatgctactcaggggctttatgaaccatccatcaattctgaagttctgactctcccattaccctttccctggtgtggtcagaactccaggtcactggaagttagtggaatcatgtagttgaattctttacttcaagacattgtattctctccagctatcaaaacattaatgatcttttatgtcttttttttgttattgttatactttaagttctggggtacatgtgcggaacatgtaggtttgttacataggtatacatgtgccatggtggtttgctgcactcatcaacctgtcatctacattcttttatgtctgtctttcaaagcaacactctgttcttctgagtagtgaaatcaggtcaactttaccaccagcctccatttttaatatgcttcaccatcatccagcacctacttaagatttatctagggctctgtggtgatgttaggacccataaaagaaatttatgccttccatatgtttggttacagatgggaaatgggaatgttgaaggacatgaaagaaaggatgtttacacattaagcatcagttctgaagctagattgtctgagtttgaatcttagctcttccctttattagctctgtgacctcgagctagttacttaaatgctctgatcctctatttcctgatcagtgaaacctccctattcaaatgtgtgagagtttaataaattaggacacttaaaaatgttggagcagtgcatagcatgtagtgttcagtacatgttaaatgttgttttttattatgtacaaacatgagtgggcacagaattttaaatcatctcaacttttgagaaattttgagttatcaacaccgttcccacaagacagtggcaaaattattggtgagaattaaacagctgtttctcagaggaagcaatggaggcttgctgggataaaggcatttactgagaggctgttacctagtgagagtgatgaattaattaaaatagtcgaatccctttctgactgtctctgaaagcttccgcttttatctttgaagagcagaattgtcactccaaggacatttattaataaaaagaacaactgtccagtgcaatgaaggcaaagtcataggtctcccaagtcttaccccattcctgtgaaatatcaagttcttggcttttctctgtcatgtagcctcaactttctctgaccgggtgcatttctttctctggtttctaaattgccagtggcaaatttggatcacttacttaatatctgttaaattttgtgacccaacaaagtcttttagcactgtggtgtcaaaaagaaaaacacctcccaggcatatacattttatagattcctggagaatgttgctctccagctccatccccacccaatgaaatatgatccagagagtcttgcaaagagacaagcctcattttccacaattagctctaaagtgcctccaggaaatgattttctcagctcatctctctgtattccctgttttggatcacagggcaatctgtttaaatgactaattacagaaatcattaaaggcaccaagcaaatgtcatctctgaatacacacatcccaagctttacaaatcctgcctggcttgacagtgatgaggccacttaacagtccagcgcaggcggatgttaaaaaaaataaaaaggtgaccatctgcggtttagttttttaactttctgatttcacacttaacgtctgtcattctgttactgggcacctgtttaaattctattttaaaatgttaatgtgtgttgtttaaaataaaatcaagaaagagaga

SEQ ID NO: 36 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 37 - protein, 277 aa, Homo sapiens [sequence omitted]

SEQ ID NO: 38 - protein, 252 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELLFHLEALSG

SEQ ID NO: 39 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 40 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 41 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 42 - protein, 9 aa, Simian virus 40
SEFLLEKRI

SEQ ID NO: 43 - protein, 9 aa, Mus musculus
SVYDFFVWL

SEQ ID NO: 44 - RNA, 21 nt, Artificial Sequence
  Synthetic
auguugcugacagaauucgac

SEQ ID NO: 45 - RNA, 21 nt, Artificial Sequence
  Synthetic
augaaucgacuaugcagugac

SEQ ID NO: 46 - RNA, 21 nt, Artificial Sequence
  Synthetic
auguuauccagcaggucgucc

SEQ ID NO: 47 - RNA, 21 nt, Artificial Sequence
  Synthetic
auagugugacauggaagagaa

SEQ ID NO: 48 - DNA, 21 bp, Homo sapiens
gtcgaattctgtcagcaacat

SEQ ID NO: 49 - DNA, 21 bp, Homo sapiens
gtcactgcatagtcgattcat

SEQ ID NO: 50 - DNA, 21 bp, Homo sapiens
ggacgacctgctggataacat

SEQ ID NO: 51 - DNA, 21 bp, Homo sapiens
ttctcttccatgtcacactat

SEQ ID NO: 52 - DNA, 2143 bp, Homo sapiens
agtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaaaaa

SEQ ID NO: 53 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 54 - DNA, 1524 bp, Homo sapiens
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 55 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 56 - DNA, 3878 bp, Homo sapiens
ggctccccactctgccagagcgaggcggggcagtgaggactccgcgacgcgtccgcaccctgcggccagagcggctttgagctcggctgcgtccgcgctaggcgctttttcccagaagcaatccaggcgcgcccgctggttcttgagcgccaggaaaagcccggagctaacgaccggccgctcggccactgcacggggccccaagccgcagaaggacgacgggagggtaatgaagctgagcccaggtctcctaggaaggagagagtgcgccggagcagcgtgggaaagaagggaagagtgtcgttaagtttacggccaacggtggattatccgggccgctgcgcgtctgggggctgcggaatgcgcgaggagaacaagggcatgcccagtgggggcggcagcgatgagggtctggccagcgccgcggcgcggggactagtggagaaggtgcgacagctcctggaagccggcgcggatcccaacggagtcaaccgtttcgggaggcgcgcgatccaggtcatgatgatgggcagcgcccgcgtggcggagctgctgctgctccacggcgcggagcccaactgcgcagaccctgccactctcacccgaccggtgcatgatgctgcccgggagggcttcctggacacgctggtggtgctgcaccgggccggggcgcggctggacgtgcgcgatgcctggggtcgtctgcccgtggacttggccgaggagcggggccaccgcgacgttgcagggtacctgcgcacagccacgggggactgacgccaggttccccagccgcccacaacgactttattttcttacccaatttcccacccccacccacctaattcgatgaaggctgccaacggggagcggcggaaagcctgtaagcctgcaagcctgtctgagactcacaggaaggaggagccgaccgggaataaccttccatacatttttttctttgtcttatctggccctcgacactcaccatgaagcgaaacacagagaagcggatttccagggatatttaggagtgtgtgacattccaggggtcgtttgcttttcagggttttctgagggaaagtgcatatgaaatccttgactggacctggtggctacgaatcttccgatggatgaatctcccactccagcgctgagtgggagaaggcagtgattagcacttgggtgacggcagtcgatgcgttcactccaatgtctgctgaggagttatggtgaacccacaacttaggccctagcggcagaaaggaaaacctgaagactgaggacaaagtggaggagggccgaggtgggcttcagtaagtccccggcggcgctttagtttgagcgcatggcaagtcacatgcgtaaacgacactctctggaagccctggagaccctcgcccaactccaccagatagcagaggggtaagagaggatgtgcaagcgacgacagatgctaaaatccctggatcacgacgctgcagagcacctttgcacaggatgctggcctttgctcttactacactgaggagagattcccgcgggttccgcaggcagactacacaggatgaggtggtggagtggagtgagagcaattgtaacggttaactgtaacgttttctttcacacacacacacacacacacacacacacatgctaggatgcggaaatccccttatgacttgctactttttgattttgtgatattttgtactttttagttgttcagcaactgtcttatttaatggggagattttaagtaacataactagtggctctcagttaaaatgtgaggaagaactacagctcttaaatgtagcaatggcactgttgcaaactcagtgcaaacgcctagattgctttcttcttaacctatttatttctttgttaaatttttctgattgtttcctttatagagtgtctcagggtgcagaggtcagactaagaaatattccaaatgtcttttagaagatagatgcacttatgcagtaaattatcttgggatagttcccaaaagattgctgaaaaagtagattgagtataaaaacttgaaaatatatgatggctcgtgggatgtcctactatcactgaacaaactaaaggtgcactgctttgggatttaatttccagggttgcttgatcattatatcattggaacaactgatacttcactactttaataaagaattaacagagattgaactccaagaggtgggtaatttggtttaaaaatacatgttcatgggtttaccactaactcctgagaaatgttaaaggttcacaggggttcccttctctcaatgtttgtaataattgctcataagcaataccagcaattcataaaaactgcttacttatgccatagaaaattaaacacaaagtgtatacatgtattatgcttctaaatgctcattctaccagatacacatttaaaagagaaaaaaggaacagaaacaagtcatttgagagtggagacttataagaaggagtacatttgagttgaatacacaaatctttacttctctaccaattcctattcccaaaatgaacatattactggggaaagttagttgagaatcagagcatatgttattggggaaaggatatgtttattgacacataatctgtaccaggtatgcattaaaatatatttgttaatttaatatttaaacctgagagataggtattgtttcccagatgaggacaatgaggcaaagaaatatcaagtaacttgccaaaggttacaagatattcattccatggatgcacaaagaagtgcatctagttccacagctgattatggttgtcttgcttttcttcccattgcaccagcttgtcctccaaaatcatgaatgatacacatgaagataactttttttaaaaaaaagcagaaatacacaatgatctcccttgtaagctcctaaggtggcttttctttctctaacttctagtaaatataaacggtttgtttgaaaactattttaaaatgtcaacaatatggagaataaccccccccaacacacctataaaaacccaaatttttggaacaaagataatggaacctccattttcaaactgaagcacagggacagaaaatatatttctagttatcacttaagcactcaatcattagaggctacaagaataatatttttaaagttacagtattttacaattattagaaaacattctatataaaagaagtcagttgatactttaaaatctcccatttggtttataaaatcccttaatttgacctctatatcttaaattccaagatgtttaaatttgctagttgcattatactgggtcatgaaaaattatcccttgaaatagatatgaaacatgttacttcatttctggtttaaataacttgtggaatctttcctaatgacaacctgatattaagggaaactaaagaaaatgttattgtggatcccacagtactatattacactgttttttttgtttgttttgttagttttttttatttaaagcaaacctcaaacattattgggtatcaattaccacctggttgtattgaaatagtaacttatcaatgccatgtaaaaattaattccattttcgaagccacctggcagacaggtttagctgtttcatcagcagcctaatatatactgttaaatttgttaaggatttcactttgaaggatacatgcaaaacatatagttactattttcatgagtcctgcttctagctccattgtggaatacagaaaattaaatatacctgttaagttcgtatctaaacctaagacattaccaaggtttgtacaaattctactacctgacatttattccaagaagatctggaaagttaaataaatttataaatttaataacaaaaaaaaaaaaaaaaaa

SEQ ID NO: 57 - protein, 138 aa, Homo sapiens
MREENKGMPSGGGSDEGLASAAARGLVEKVRQLLEAGADPNGVNRFGRRAIQVMMMGSARVAELLLLHGAEPNCADPATLTRPVHDAAREGFLDTLVVLHRAGARLDVRDAWGRLPVDLAEERGHRDVAGYLRTATGD

SEQ ID NO: 58 - DNA, 1474 bp, Homo sapiens
agccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaaaaaaaaaaaaaaaaaaaaaaaaaaaaaaaa

SEQ ID NO: 59 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 60 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 61 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 62 - DNA, 1755 bp, Homo sapiens
agagagaagcgaggtttccattctgagggacggcgtagagttcggccgaaggaacctgacccaggctctgtgaggaggcaaggttttcaggggacaggccaacccagaggacaggattccctggaggccacagaggagcaccaaggagaagatctgcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttaaaaaaaaaaaaaaaaaaaaaaaaaaaaaaaaaaaaaaaaaaaaaa

SEQ ID NO: 63 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 64 - DNA, 2495 bp, Homo sapiens
gcggctgcggggttcctggtgctgaggacggacgccattggagttcccgagaagcatggctgagggaagcttcagcgtgcaatcggaaagctacagtgttgaagacatggatgagggtagcgacgaagtcggggaggaagagatggttgaaggcaacgactatgaagaattcggtgcgtttggtggctatggcaccctcaccagctttgacatccatatcctcagagccttcggaagcttgggtccaggccttcgcatcttatcgaatgagccctgggaactggaaaaccctgtgctggcccagaccctggtggaggcattgcagctggatccggaaacacttgccaatgagacggccgcccgtgctgccaacgtagcccgcgccgccgcctccaaccgtgcggctcgggccgctgccgccgctgcccgtaccgccttcagtcaggtggtcgctagccaccgggtggccacgccgcaggtctcaggagaggatacccagcccacgacctacgccgccgaggctcaggggcccacccctgagccaccccttgcttctccgcagacctcccagatgttagtcaccagtaagatggctgcccccgaggctccggcaacctccgcacagtcccagacaggctccccggcccaggaggctgctactgagggccctagtagcgcctgtgctttctctcaggctccgtgtgccagggaggtggacgccaaccggcccagcacagccttcctgggccagaatgatgtcttcgatttcactcagccggcaggtgtcagtggcatggccttcccgcgccccaagagacctgccccagcccaagaggctgccacagagggccccagtgctgcctctggtgtgccccagacgggacctggcagggaggtggcagccacccggcccaagaccaccaagtcggggaaggcgctggccaagactcggtgggtggagcctcagaatgttgtggcagcagctgctgccaaggccaagatggccacgagcatccctgagccggagggtgcagctgctgccactgctcagcacagtgctgagccctgggccaggatgggaggcaagaggaccaagaagtccaagcacctggatgatgagtatgagagcagcgaggaggagagagagactcccgcggtcccacccacctggagagcatcacagccctcattgacggtgcgggctcagttggcccctcggcccccgatggccccgaggtcccagataccctcaaggcacgtactgtgcctgcccccccgcaacgtgacccttctgcaggagagggcaaataagttggtgaaatacctgatgattaaggactacaagaagatccccatcaagcgcgcagacatgctgaaggatgtcatcagagaatatgatgaacatttccctgagatcattgaacgagcaacgtacaccctggaaaagaagtttgggatccacctgaaggagatcgacaaggaagaacacctgtatattcttgtctgcacacgggactcctcagctcgcctccttggaaaaaccaaggacactcccaggctgagtctcctcttggtgattctgggcgtcatcttcatgaatggcaaccgtgccagcgaggctgtcctctgggaggcactacgcaagatgggactgcgccctggggtgaggcacccattcctcggcgatctgaggaagctcatcacagatgactttgtgaagcagaagtacctggaatacaagaagatccccaacagcaacccacctgagtatgaattcctctggggcctgcgagcccgccatgagaccagcaagatgagggtcctgagattcatcgcccagaatcagaaccgagacccccgggaatggaaggctcatttcttggaggctgtggatgatgctttcaagacaatggatgtggatatggccgaggaacatgccagggcccagatgagggcccagatgaatatcggggatgaagcgctgattggacggtggagctgggatgacatacaagtcgagctcctgacctgggatgaggacggagattttggcgatgcctgggccaggatcccctttgctttctgggccagataccatcagtacattctgaatagcaaccgtgccaacaggagggccacgtggagagctggcgtcagcagtggcaccaatggaggggccagcaccagcgtcctagatggccccagcaccagctccaccatccggaccagaaatgctgccagagctggcgccagcttcttctcctggatccagtaagagtttcggcaccgttgacgaactgcagcgatcttactggccaagccagagcgcctcctctcagattccttctcgacacagcaccctaggcggcttcttcctgtcagtcggaggtggcatgcaagatgaagctctctttgctcttcctgctttcattttgtgcttttccttgtgttttcatgttttgggtatcagtgttacattaaagttgcaaaattaa

SEQ ID NO: 65 - protein, 739 aa, Homo sapiens
MAEGSFSVQSESYSVEDMDEGSDEVGEEEMVEGNDYEEFGAFGGYGTLTSFDIHILRAFGSLGPGLRILSNEPWELENPVLAQTLVEALQLDPETLANETAARAANVARAAASNRAARAAAAAARTAFSQVVASHRVATPQVSGEDTQPTTYAAEAQGPTPEPPLASPQTSQMLVTSKMAAPEAPATSAQSQTGSPAQEAATEGPSSACAFSQAPCAREVDANRPSTAFLGQNDVFDFTQPAGVSGMAFPRPKRPAPAQEAATEGPSAASGVPQTGPGREVAATRPKTTKSGKALAKTRWVEPQNVVAAAAAKAKMATSIPEPEGAAAATAQHSAEPWARMGGKRTKKSKHLDDEYESSEEERETPAVPPTWRASQPSLTVRAQLAPRPPMAPRSQIPSRHVLCLPPRNVTLLQERANKLVKYLMIKDYKKIPIKRADMLKDVIREYDEHFPEIIERATYTLEKKFGIHLKEIDKEEHLYILVCTRDSSARLLGKTKDTPRLSLLLVILGVIFMNGNRASEAVLWEALRKMGLRPGVRHPFLGDLRKLITDDFVKQKYLEYKKIPNSNPPEYEFLWGLRARHETSKMRVLRFIAQNQNRDPREWKAHFLEAVDDAFKTMDVDMAEEHARAQMRAQMNIGDEALIGRWSWDDIQVELLTWDEDGDFGDAWARIPFAFWARYHQYILNSNRANRRATWRAGVSSGTNGGASTSVLDGPSTSSTIRTRNAARAGASFFSWIQ

SEQ ID NO: 66 - DNA, 1753 bp, Homo sapiens
gagattctcgccctgagcaacgagcgacggcctgacgtcggcggagggaagccggcccaggctcggtgaggaggcaaggttctgaggggacaggctgacctggaggaccagaggcccccggaggagcactgaaggagaagatctgccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcaaaaaaaaaaaaaaaaaaaaaaaaaaaaaa

SEQ ID NO: 67 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 68 - DNA, 2126 bp, Homo sapiens
gccaggctctccacccccacttcccaattgaggaaaccgaggcagaggaggctcagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgtaaaaaaaaaaaaaaaaaa

SEQ ID NO: 69 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 70 - DNA, 2082 bp, Homo sapiens
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttattttaaaaaattgaaataattttgatttttgccttctgattatttaaagatctatatatgttttattggccccttctttattttaataaaacagtgagaaatctaaaaaaaaaaaaaaaaaa

SEQ ID NO: 71 - protein, 529 aa, Homo sapiens [sequence omitted]

SEQ ID NO: 72 - DNA, 2876 bp, Homo sapiens
acagttctgacgtgagaggacaagaaacacaagcaaatataaaacattcaattctaagagaagttcatcagagacatccttcaggattgtgagctggattttcctctacgtgcttcagtcttctctacacaaagagctgcaaaccaggtctttgttttgcactcttatttcaagcagaatgagtgctcctaaactcctctctctgggctgtatcttcttccccttgctactttttcagcaggcccgggctcaattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggaaatgttgcaagagccttctttctcccttccttactggaattttgcaacggggaaaaatgtctgtgatatctgcacggatgacttgatgggatccagaagcaactttgattccactctaataagcccaaactctgtcttttctcaatggcgagtggtctgtgactccttggaagattatgataccctgggaacactttgtaacagcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctgatatatccacatttccattggaaaatgcccctattggacataatagacaatacaacatggtgccattctggcccccagtcaccaacacagaaatgtttgttactgctccagacaacctgggatacacttatgaaattcaatggccaagtcgggagtttagtgtacctgagataattgccatagcagtagttggcgctttgttactggttgcactcatttttgggactgcttcttatctgattcgtgccagacgcagtatggatgaagctaaccagcctctcctcactgatcagtatcaatgctatgctgaagaatatgaaaaactccagaatcctaatcagtctgtggtctaacaaatgccctactctcttatgcattagtatcacaaaaccacctggttgaatataatagattgagttattaactgtattttctttcactttattaccttctttctaatacaagcatatgttagcattaaagttctaggcatacttttcaaagctgggaagaccctttcagaatcttttcaatgggttttaattttcagttctatttaaaatggtgaatgacactaaactccatgatatttaaggatagtgtgaagatctttggcatgatttaaaggttgagtatgtgaagatataagtaagtgaactaccatgctttgtttacgtgtaaaggaaaataatgtttgatagtaaatgtccacttaaaatacatgaatgggcatttctaaaatgttaaaacataaacacatttccattcatggatatttgtcaacagatttaaagaaaaccacagttattaattaaagaaaattaattatgtgtagttataaaccaatgaaattttgattaaccttttcaaattaatgttccagtttgaagaccaatcaaatatattatttagtcaacatatactatttagtctcaggttcaaggctacaacaaaaatcaccatctttgtcaaactttggagagggaaaatcttcactttcttaagcaacaatggatattgcctgtgtttgccactgtgtttccctgcctctcaattcgctgaaaaaggaactacctatccttacatttcacctactaatgtctcttctaacatcttagaggtccatggagaaggcatatggagaacatgttttatactgctctataaatagtattccaatcactgtgcttaatttaaatagcattatcttatcatttatcagccttttatgtattttccaagtaaaatattaacatattatttcattggtcttcttttttatctggttctatatgaatgctattttttcccttctcttctaacatgaaatatattttctctttttgatcttgtgctatgaaacaatcttccaaagaactgtataaggtggtcataagtgaatattttaattaaaattggtaaaaataaataataaca

SEQ ID NO: 73 - protein, 537 aa, Homo sapiens
MSAPKLLSLGCIFFPLLLFQQARAQFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 74 - DNA, 2403 bp, Homo sapiens
aactgagttcaaggcaattaaagtcaagagctaaggagggagggagagggtttagaaataccagcataataagtagtatgactgggtgctctgtaaattaactcaattagacaaagcctgacttaacgggggaagatggtgagaagcgctaccctcattaaatttggttgttagaggcgcttctaaggaaattaagtctgttagttgtttgaatcacataaaattgtgtgtgcacgttcatgtacacatgtgcacacatgtaacctctgtgattcttgtgggtatttttttaagaagaaaggaatagaaagcaaagaaaaataaaaaatactgaaaagaaaagactgaaagagtagaagataaggagaaaagtacgacagagacaaggaaagtaagagagagagagagctctcccaattataaagccatgagccccctttggtgggggtttctgctcagttgcttgggctgcaaaatcctgccaggagcccagggtcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggtgatttctaatcgtttgctttacaatgctacaacaaacatccttgaacatgtaagaaaagagaaagcgaccaaggaactcccttccctgcatgtgctggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactctcttagtagtcatgggaacactggtggctttggttggtctttttgtgctgttggcttttcttcaatatagaagacttcgaaaaggatatacacccctaatggagacacatttaagcagcaagagatacacagaagaagcctagggtgctcatgccttacctaagagaagaggctggccaagccacagttctgacgctgacaataaaggaactaatcctcactgttccttcttgagttgaagatctttgacataggttcttctatagtgatgatgatctcattcagaagatgcttagctgtagtttccgctttgcttgcttgtttaacaaacccaactaaagtgcttgaggctacctctaccttcaaataaagatagacctgacaatttgtgatatctaataataaccccccccccaatattgattaagcctcctccttttctgaaagcatttaaaaaaaa

SEQ ID NO: 75 - protein, 552 aa, Homo sapiens
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVISNRLLYNATTNILEHVRKEKATKELPSLHVLVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 76 - DNA, 1374 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagtcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 77 - protein, 356 aa, Homo sapiens [sequence omitted]

(19) United States
(12) Patent Application Publication
(10) Pub. No.: US 2013/0022640 A1
(43) Pub. Date: Jan. 24, 2013

(54) IMMUNE MODULATORS RELATING TO FOXO3A

(71) Applicants: Arthur A. Hurwitz, N. Potomac, MD (US); Stephanie K. Watkins, Middletown, MD (US)

(72) Inventors: Arthur A. Hurwitz, N. Potomac, MD (US); Stephanie K. Watkins, Middletown, MD (US)

(73) Assignees: Secretary, Department of Health and Human Services, Bethesda, MD (US); The United States of America, as represented by the

(21) Appl. No.: 13/520,904

(22) PCT Filed: Jan. 6, 2011
(86) PCT No.: PCT/US11/20315

§ 371 Date: Jul. 13, 2012

Related U.S. Application Data
(60) Provisional application No. 61/293,098, filed on Jan. 7, 2010.

(51) Int. Cl. A61K 39/00; A61P 35/00; A61P 37/06; A61K 35/12

(52) U.S. Cl. 424/277.1; 424/93.7; 424/93.21; 424/184.1

(57) ABSTRACT

The invention provides methods of enhancing an immune response to a cancer antigen in a mammal comprising inhibiting the activity of the foxo3a gene or gene product in dendritic cells in the mammal. The invention also provides methods of suppressing an immune response to an autoimmune disease antigen in a mammal comprising increasing the activity of the foxo3a gene or gene product in dendritic cells in the mammal. The invention also provides related methods of treating cancer and autoimmune diseases.

### CROSS-REFERENCE TO RELATED APPLICATION

This patent application claims the benefit of U.S. Provisional Patent Application No. 61/293,098, filed Jan. 7, 2010, which is incorporated by reference.

### BACKGROUND OF THE INVENTION

T cell tolerance often plays an important role in the progression of disease and the effectiveness of medical treatments for disease. For example, the successful treatment of some diseases using immunotherapy can be limited by T cell tolerance. This is thought to be true, for example, in the treatment of cancer. T cell tolerance disadvantageously results in the loss of antigen-specific T cell function and the failure of the T cell to immunologically respond to a disease antigen and/or the failure to cause killing of the cell presenting the disease antigen. This T cell tolerance is thought to be responsible, at least in part, for the progression of the disease and reduction in the effectiveness of some treatments, particularly immunotherapy treatments.

Conversely, in the context of other diseases, the loss of T cell tolerance is undesirable and at least partly responsible for the progression of the disease or reduction in the effectiveness of treatment. Autoimmune diseases, for example, are caused at least in part by the loss of T cell tolerance for “self” antigens, which leads to the destruction of “self” tissues.

Thus, there is a need for methods of modulating an immune response.

### BRIEF SUMMARY OF THE INVENTION

An embodiment of the invention provides a method of enhancing an immune response to a cancer antigen in a mammal, which method comprises administering a cancer antigen to a mammal and inhibiting the activity of the foxo3a gene or gene product in dendritic cells in the mammal by administering an siRNA selected from the group consisting of (a) SEQ ID NOs: 44-47 and 1-4 and (b) siRNAs having at least 95% identity to any one of SEQ ID NOs: 44-47 and 1-4 and a nucleotide length of about 18 to about 30 to the mammal, thereby enhancing an immune response to the cancer antigen in the mammal.

Another embodiment of the invention provides a method of enhancing an immune response to a cancer antigen in a mammal, which method comprises (a) obtaining dendritic cells from the mammal; (b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells; (c) inhibiting the activity of the foxo3a gene or gene product in the dendritic cells by delivering an siRNA selected from the group consisting of (i) SEQ ID NOs: 44-47 and 1-4 and (ii) siRNAs having at least 95% identity to any one of SEQ ID NOs: 44-47 and 1-4 and a nucleotide length of about 18 to about 30 to the dendritic cells to produce dendritic cells having decreased foxo3a gene or gene product activity; and (d) administering the antigen-expressing dendritic cells having decreased foxo3a gene or gene product activity to the mammal, thereby enhancing an immune response to the cancer antigen in the mammal.

Still another embodiment of the invention provides a method of enhancing an immune response to a cancer antigen in a mammal, which method comprises (a) obtaining T cells and dendritic cells from the mammal; (b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells; (c) inhibiting the activity of the foxo3a gene or gene product in the dendritic cells by delivering an siRNA selected from the group consisting of (i) SEQ ID NOs: 44-47 and 1-4 and (ii) siRNAs having at least 95% identity to any one of SEQ ID NOs: 44-47 and 1-4 and a nucleotide length of about 18 to about 30 to the dendritic cells to produce dendritic cells having decreased foxo3a gene or gene product activity; (d) exposing the antigen-expressing dendritic cells having decreased foxo3a gene or gene product activity to the T cells; and (e) administering the T cells to the mammal, thereby enhancing an immune response to the cancer antigen in the mammal.

Another embodiment of the invention provides a method of suppressing an immune response to an autoimmune disease antigen in a mammal, which method comprises administering the autoimmune disease antigen to the mammal and increasing the activity of the foxo3a gene or gene product in dendritic cells in the mammal by administering a nucleic acid encoding foxo3a selected from the group consisting of (i) SEQ ID NOs: 5-6 and (ii) sequences having at least 95% identity to SEQ ID NO: 5 or 6 to the mammal, thereby suppressing an immune response to the autoimmune disease antigen in the mammal.

An embodiment of the invention provides a method of suppressing an immune response to an autoimmune disease antigen in a mammal, which method comprises (a) obtaining dendritic cells from the mammal; (b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells; (c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity; and (d) administering the antigen-expressing dendritic cells having increased foxo3a gene or gene product activity to the mammal, thereby suppressing an immune response to the autoimmune disease antigen in the mammal.

Still another embodiment of the invention provides a method of suppressing an immune response to an autoimmune disease antigen in a mammal, which method comprises (a) obtaining T cells and dendritic cells from the mammal; (b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells; (c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity; (d) exposing the antigen-expressing dendritic cells having increased foxo3a gene or gene product activity to the T cells; and (e) administering the T cells to the mammal, thereby suppressing an immune response to the autoimmune disease antigen in the mammal.

### BRIEF DESCRIPTION OF THE SEVERAL VIEWS OF THE DRAWING(S)

FIG. 1A is a graph showing proliferation as indicated by counts per minute (CPM) (y axis) of naïve TcR-I T cells stimulated with various concentrations of SV40 T prostate tumor antigen (TAg) (x axis) and wild-type (WT) dendritic cells (DCs) (black squares) or TRansgenic Adenocarcinoma of the Mouse Prostate (TRAMP) DCs (black triangles). *p<0.0001 WT vs. TRAMP (Student's t test). Data are representative of 4 independent trials (3 WT and 3 TRAMP mice in each experiment), mean±standard deviation (s.d.).

FIG. 1B is a graph showing proliferation as indicated by CPM (y axis) of naïve TcR-I T cells co-cultured with WT DCs (black squares) or TRAMP DCs (black triangles) for four days prior to secondary stimulation with various concentrations of TAg (x axis) and splenic APCs. *p<0.0001 WT vs. TRAMP (Student's t test). Data are representative of 4 independent trials (3 WT and 3 TRAMP mice in each experiment), mean±s.d.

FIG. 2A is a graph showing interferon-gamma (IFN-γ) secretion as measured by spots per 10,000 effector cells (y axis) by naïve TcR-I T cells co-cultured with WT DCs (black squares) or TRAMP DCs (black triangles) for four days prior to secondary stimulation with various concentrations of TAg (x axis) and splenic APCs. *p<0.0001 WT vs. TRAMP (Student's t test). Data are representative of 2 independent trials (3 WT and 3 TRAMP mice in each experiment), mean±s.d.

FIG. 2B is a graph showing proliferation as indicated by CPM (y axis) of effector TcR-I T cells co-cultured with WT DCs (black squares) or TRAMP DCs (black triangles) for four days prior to secondary stimulation with various concentrations of TAg (x axis) and splenic APCs. *p<0.001 WT vs. TRAMP (Student's t test). Data are representative of 2 independent trials (3 WT and 3 TRAMP mice in each experiment), mean±s.d.

FIG. 3A is a graph showing granzyme B secretion as measured by spots per 5,000 cells (y axis) by T cells re-isolated and stimulated with various concentrations of TAg (x axis) 6 days after transfer into TRAMP mice with (black triangles) or without (black circles) TADC depletion. Data are representative of 3 independent trials with 3-5 mice per group, mean±s.d., p<0.001, **p<0.0001 (Student's t-test).

FIG. 3B is a graph showing IFN-γ secretion as measured by spots per 10,000 cells (y axis) by T cells re-isolated and stimulated with various concentrations of TAg (x axis) 6 days after transfer into WT mice without TADC depletion (black squares) or TRAMP mice with (black triangles) or without (black circles) TADC depletion. Data are representative of 3 independent trials with 3-5 mice per group, mean±s.d., p<0.001, p<0.0001 (Student's t-test).

FIG. 3C is a graph showing granzyme B secretion as measured by spots per 5,000 cells (y axis) by T cells re-isolated and stimulated with various concentrations of TAg (x axis) 12 days after transfer into TRAMP mice with (black triangles) or without (black circles) TADC depletion. Data are representative of 3 independent trials with 3-5 mice per group, mean±s.d., p<0.001, *p<0.0001 (Student's t-test).

FIG. 3D is a graph showing IFN-γ secretion as measured by spots per 10,000 cells (y axis) by T cells re-isolated and stimulated with various concentrations of TAg (x axis) 12 days after transfer into WT mice without TADC depletion (black squares) or TRAMP mice with (black triangles) or without (black circles) TADC depletion. Data are representative of 3 independent trials with 3-5 mice per group, mean±s.d., *p<0.001, p<0.0001 (Student's t-test).

FIG. 4A is a graph showing the weight in grams (y axis) of the urogenital tract (UGT) dissected from TRAMP mice that were administered control antibody only (TRAMP), TcR-1 cells and control antibody (TRAMP+TcR-I), α-CD317 (TRAMP+α-CD317), or both TcR-1 cells and α-CD317 (TRAMP+α-CD317+ TcR-I). Mean±S.E.M., *p<0.01 (Student's t-test).

FIG. 4B is a graph showing the weight in grams (y axis) of the prostate dissected from TRAMP mice that were administered control antibody only (TRAMP), TcR-1 cells and control antibody (TRAMP+TcR-I), α-CD317 (TRAMP+α-CD317), or both TcR-1 cells and α-CD317 (TRAMP+α-CD317+ TcR-I). Mean±S.E.M., *p<0.01 (Student's t-test).

FIG. 5A is a graph showing IFN-γ secretion as measured by spots per 10,000 cells (y axis) by TcR-I T cells re-isolated and stimulated with various concentrations of TAg (x axis) 6 days after transfer into WT mice (black diamonds), untreated TRAMP mice (black circles), or TRAMP mice that were previously treated with 1-methyl-D-tryptophan (1MDT) (black triangles) or (S)-(2-boronoethyl)-L-cysteine (BEC) (black squares). Data are representative of 3 independent trials with 3-5 mice per group, mean±s.d., *p<0.05 (Student's t-test).

FIG. 5B is a graph showing granzyme B secretion as measured by spots per 5,000 cells (y axis) by TcR-I T cells re-isolated and stimulated with various concentrations of TAg (x axis) 6 days after transfer into untreated TRAMP mice (black circles) or TRAMP mice that were previously treated with 1 MDT (black triangles) or BEC (black squares). Data are representative of 3 independent trials with 3-5 mice per group, mean±s.d., **p<0.001 (Student's t-test).

FIG. 5C is a graph showing IFN-γ secretion as measured by spots per 10,000 cells (y axis) by TcR-I T cells re-isolated and stimulated with various concentrations of TAg (x axis) 6 days after transfer into WT mice previously treated with control antibody (Ab) (black diamonds), TRAMP mice previously treated with control Ab (black circles), or TRAMP mice that were previously treated with anti-PD-1 Ab (black squares). Data are representative of 3 independent trials with 3-5 mice per group, mean±s.d., p<0.05 (Student's t-test).

FIG. 5D is a graph showing granzyme B secretion as measured by spots per 5,000 cells (y axis) by TcR-I T cells re-isolated and stimulated with various concentrations of TAg (x axis) 6 days after transfer into TRAMP mice previously treated with control Ab (black circles) or anti-PD-1 Ab (black squares). Data are representative of 3 independent trials with 3-5 mice per group, mean±s.d., *p<0.05 (Student's t-test).

FIG. 5E is a graph showing IFN-γ secretion as measured by spots per 10,000 cells (y axis) by TcR-I T cells re-isolated and stimulated with various concentrations of TAg (x axis) 6 days after transfer into WT mice previously treated with control Ab (black diamonds), TRAMP mice previously treated with control Ab (black circles), or TRAMP mice that were previously treated with anti-TGF-β Ab (black squares). Data are representative of 3 independent trials with 3-5 mice per group, mean±s.d.

FIG. 5F is a graph showing granzyme B secretion as measured by spots per 5,000 cells (y axis) by TcR-I T cells re-isolated and stimulated with various concentrations of TAg (x axis) 6 days after transfer into TRAMP mice previously treated with control Ab (black circles) or anti-TGF-β Ab (black squares). Data are representative of 3 independent trials with 3-5 mice per group, mean±s.d., *p<0.05, **p<0.001 (Student's t-test).

FIG. 6A is a graph showing proliferation as indicated by CPM (y axis) of TcR-I T cells co-cultured with various concentrations of TAg (x axis) and WT DCs (black diamonds), TRAMP DCs (black circles), WT DCs and 1MDT (white diamonds), or TRAMP DCs and 1MDT (white circles). Data is representative of 3 independent trials of 3-5 mice per group, mean±s.d., *p<0.05 (Student's t-test).

FIG. 6B is a graph showing proliferation as indicated by CPM (y axis) of TcR-I T cells co-cultured with various concentrations of TAg (x axis) and WT prostate DCs (black diamonds), TRAMP DCs (black circles), WT prostate DCs and anti-PD-1 Ab (white diamonds), or TRAMP DCs and anti-PD-1 Ab (white circles). Data is representative of 3 independent trials of 3-5 mice per group, mean±s.d., *p<0.05 (Student's t-test).

FIG. 6C is a graph showing proliferation as indicated by CPM (y axis) of TcR-I T cells co-cultured with various concentrations of TAg (x axis) and WT DCs (black diamonds), TRAMP DCs (black circles), WT DCs and anti-TGF-β Ab (white diamonds), or TRAMP DCs and anti-TGF-(3 Ab (white circles). Data is representative of 3 independent trials of 3-5 mice per group, mean±s.d., *p<0.05 (Student's t-test).

FIG. 6D is a graph showing proliferation as indicated by CPM (y axis) of TcR-I T cells co-cultured with WT DCs (white squares), TRAMP DCs (black circles), TRAMP DCs and BEC (black squares), TRAMP DCs and 1MDT (black triangles), TRAMP DCs and anti-PD-1 Ab (black diamonds), or TRAMP DCs and anti-TGF-β Ab (white circles) for four days prior to secondary stimulation with various concentrations of TAg (x axis) and splenic APC. *p<0.0001 WT vs. TRAMP (Student's t test). Data are representative of 4 independent trials (3 WT and 3 TRAMP mice in each experiment), mean±s.d.

FIG. 7A is a graph showing UGT weight in grams (y axis) 12 days after transfer of TcR-I cells into untreated TRAMP mice (TRAMP), or TRAMP mice treated with BEC, 1MDT, anti-TGF-β Ab, or anti-PD-1 Ab (x axis). Dashed lines represent the average WT weight. Data are presented for two combined studies with 7 mice total per treatment group, mean±S.E.M. *p<0.05, **p<0.001 (Student's t-test).

FIG. 7B is a graph showing prostate weight in grams (y axis) 12 days after transfer of TcR-I cells into untreated TRAMP mice (TRAMP), or TRAMP mice treated with BEC, 1MDT, anti-TGF-β Ab, or anti-PD-1 Ab (x axis). Dashed lines represent the average WT weight. Data are presented for two combined studies with 7 mice total per treatment group, mean±S.E.M. *p<0.05, **p<0.001 (Student's t-test).

FIG. 7C is a graph showing granzyme B secretion as measured by spots per 5,000 cells (y axis) by TcR-I T cells re-isolated and stimulated with various concentrations of TAg (x axis) 6 days after transfer into untreated TRAMP mice (black circles) or TRAMP mice previously treated with anti-PD-1 Ab (black squares), 1 MDT (black triangle), or both anti-PD-1 Ab and 1MDT (white diamond). Data are representative of 2 independent trials with 3-5 mice per group, mean±s.d.

FIG. 7D is a graph showing IFN-γ secretion as measured by spots per 10,000 cells (y axis) by TcR-I T cells re-isolated and stimulated with various concentrations of TAg (x axis) 6 days after transfer into untreated TRAMP mice (black circles) or TRAMP mice previously treated with anti-PD-1 Ab (black squares), 1MDT (black triangle), or both anti-PD-1 Ab and 1MDT (white diamond). Data are representative of 2 independent trials with 3-5 mice per group, mean±s.d.

FIG. 8A is a graph showing proliferation as indicated by CPM (y axis) of naïve TcR-I cells stimulated with various concentrations of TAg (x axis) and WT DCs that were cultured with control (poorly stimulatory) siRNAs (black squares) or TRAMP DCs that were cultured with control siRNAs (black circles), foxo3a siRNA for 24 hours (black triangles ▴), or foxo3a siRNA for 48 hours (upside-down triangles ▾). *p<0.0001 WT vs. TRAMP (Student's t test). Data are representative of 3 independent experiments (3 mice per group), mean±s.d., *p<0.01 (Student's t-test).

FIG. 8B is a graph showing IFN-γ secretion as measured by spots per 10,000 effector cells (y axis) by of naïve CD8+ T cells co-cultured with WT DCs that were cultured with control siRNAs (black squares) or TRAMP DCs that were cultured with control siRNAs (black circles), foxo3a siRNA for 24 hours (black triangles ▴), or foxo3a siRNA for 48 hours (upside-down triangles ▾) for four days prior to secondary stimulation with various concentrations of TAg (x axis) and splenic APC. Data are representative of 3 independent experiments (3 mice per group), mean±s.d., *p<0.01 (Student's t-test).

FIG. 8C is a graph showing proliferation as indicated by CPM (y axis) of naïve CD8+ T cells co-cultured with WT DCs that were cultured with control siRNAs (black squares) or TRAMP DCs that were cultured with control siRNAs (black circles), foxo3a siRNA for 24 hours (black triangles ▴), or foxo3a siRNA for 48 hours (upside-down triangles ▾) for four days prior to secondary stimulation with various concentrations of TAg (x axis) and splenic APC. Data are representative of 3 independent experiments (3 mice per group), mean±s.d., *p<0.01 (Student's t-test).

FIG. 9 is a graph showing proliferation as indicated by CPM (y axis) of naïve CD8+ T cells stimulated with various concentrations of TAg (x axis) and splenic APCs and co-cultured with WT DCs (black squares), TRAMP DCs (black circles), or TRAMP DCs from mice that had previously been administered antigen-specific CD4+ (TcR-II) T cells (white triangles). Data are representative of 3 independent experiments (3 mice per group), mean±s.d., **p<0.0001 (Student's t-test).

### DETAILED DESCRIPTION OF THE INVENTION

Forkhead box O3a (foxo3a) (also known as foxo3 or FKHRL1) belongs to the forkhead family of transcription factors which are characterized by a forkhead domain. It has been discovered that certain foxo3a small interfering RNAs (siRNAs) can enhance an antigen-specific immune response. Thus, the invention provides methods of using foxo3a siRNA to enhance an immune response in a mammal.

In one embodiment, the invention provides a method of enhancing an immune response to a cancer antigen in a mammal. The method comprises administering a cancer antigen to a mammal and inhibiting the activity of the foxo3a gene or gene product in dendritic cells in the mammal by administering an siRNA selected from the group consisting of (a) SEQ ID NOs: 1-4 and 44-47 and (b) siRNAs having at least 95% identity to any one of SEQ ID NOs: 1-4 and 44-47 and a nucleotide length of about 18 to about 30 to the mammal, thereby enhancing an immune response to the cancer antigen in the mammal.

The method comprises inhibiting the activity of the foxo3a gene or gene product in dendritic cells in the mammal by administering a foxo3a siRNA to the mammal. The foxo3a siRNA may be any suitable siRNA that inhibits the activity of the foxo3a gene or gene product in a dendritic cell. The foxo3a siRNA can be a nucleic acid that specifically binds to and is complementary to a target nucleic acid encoding foxo3a or a complement thereof. The foxo3a siRNA may be introduced into a dendritic cell, wherein the dendritic cell is capable of expressing foxo3a, in an effective amount for a time and under conditions sufficient to interfere with expression of foxo3a.

The foxo3a siRNA can comprise overhangs. That is, not all nucleotides need bind to the target sequence. In some embodiments, the foxo3a siRNA comprises exclusively RNA, that is, only ribonucleic acid nucleotides. The foxo3a siRNA can also comprise DNA, that is, deoxyribonucleic acid nucleotides. The foxo3a siRNAs employed can have a length of 18 nucleotides or more, e.g., 19 nucleotides or more, 20 nucleotides or more, or 21 nucleotides or more. Alternatively, or in addition, the foxo3a siRNAs can have a length of 30 nucleotides or less, e.g., 28 nucleotides or less, 25 nucleotides or less, 24 nucleotides or less, or 22 nucleotides or less. Thus, the foxo3a siRNAs can have a length bounded by any two of the above endpoints. For example, the foxo3a siRNAs can have a length of 18-30 nucleotides, 20-25 nucleotides, or 20-22 nucleotides.

Administering or delivering an siRNA, as used herein, may include administering or delivering any nucleic acid (e.g., DNA) sequence that encodes the siRNA. In this regard, the DNA sequence encoding the foxo3a siRNA can be included in a cassette, e.g., a larger nucleic acid construct such as an appropriate vector (e.g., a recombinant expression vector). Examples of such vectors include lentiviral and adenoviral vectors, as well as other vectors described herein with respect to other aspects of the invention. An example of a suitable vector is described in Aagaard et al., Mol. Ther., 15(5): 938-45 (2007). When present as part of a larger nucleic acid construct, the nucleic acid can be longer than the DNA sequence encoding the foxo3a siRNA nucleic acid, e.g., greater than about 70 nucleotides in length.

In accordance with the invention, the foxo3a siRNA can target a nucleotide sequence of a foxo3a gene or mRNA encoded by the same. In an embodiment, the foxo3a sequence is a human sequence. For example, human foxo3a is assigned Gene NCBI Entrez Gene ID No. 2309, and an Online Mendelian Inheritance in Man (OMIM) No. 602681. The human foxo3a gene is found on chromosome 6 at 6q21. Two transcriptional variants include mRNAs: NM_{—}001455.3 and NM_{—}201559.2. Accordingly, NM_{—}001455.3 is provided as SEQ ID NO: 7, and NM_{—}201559.2 is provided as SEQ ID NO: 8. NM_{—}001455.3 and NM_{—}201559.2 encode the human FOXO3A protein sequence SEQ ID NO: 9 (corresponding to NP_{—}001446.1 or NP 963853.1). Human genomic foxo3a sequences include AJ001590.1, AL096818.9, AL365509.8, AL391646.12, and CI1471051.2. Human foxo3a mRNA sequences include AB072905.1, AF032886.1, AI554317.1, AJ001589.1, AK024103.1, AK092357.1, AK122861.1, AK301304.1, AK303933.1, BCO20227.1, BCO21224.2, BC045800.1, BC068552.1, CA389775.1, CD101760.1, CR623727.1, and CR749261.1. Human FOXO3A amino acid sequences include CAA04861.1, CAH6295.1, CAH6405.1, EAW48373.1, EAW48374.1, AAC39592.1, CAA04860.1, BAG62858.1, BAG64861.1, AAH20227.1, AAH21224.1, AAH68552.1, and CAH18117.1. Other human foxo3a species can also be employed in accordance with the invention.

In an embodiment, the foxo3a sequence is a mouse sequence. For example, mouse foxo3a is assigned Gene NCBI Entrez Gene ID No. 56484. The mouse foxo3a gene is found on chromosome 10 at 10 B2. Mouse foxo3a mRNA corresponds to the sequence NM_{—}019740.2, with corresponding protein sequence NP_{—}062714.1. Accordingly, NM_{—}019740.2 is provided as SEQ ID NO: 10, and NP_{—}062714.1 is provided as SEQ ID NO: 11. Mouse genomic foxo3a sequences include AC116179.6, AC140402.2, and CH466540.1. Mouse foxo3a mRNA sequences include AF114259.1, AK004198.1, AK008417.1, AK041595.1, AK047413.1, AK047922.1, AK079567.1, AK139629.1, AK143198.1, AK159465.1, and BC019532.1. Mouse FOXO3A amino acid sequences include EDL04998.1, AAD42107.1, BAE20596.1, BAC33049.1, BAE35106.1, AAH19532.1, and AAI66015.1. Other mouse foxo3a species can also be employed in accordance with the invention.

The foxo3a siRNA sequences can be designed against any appropriate foxo3a DNA or mRNA sequence. In this regard, the sequences of the foxo3a siRNA can be designed against a human foxo3a having a nucleotide sequence corresponding to Accession No. NM_{—}001455.3 with SEQ ID NO: 7 or Accession No. NM_{—}201559.2 with SEQ ID NO: 8. Alternatively, the sequences of the foxo3a siRNA can be designed against a mouse foxo3a having a nucleotide sequence corresponding to Accession No. NM_{—}019740.2 with SEQ ID NO: 10. In an embodiment of the invention, the foxo3a siRNA targets a foxo3a sequence selected from the group consisting of SEQ ID NOs: 12-15 and 48-51, complements thereof, and any combination thereof. The siRNA sequences of an embodiment of the invention are set forth in Table I along with their target DNA and mRNA sequences. In an embodiment, the foxo3a siRNA comprises, consists, or consists essentially of any of the siRNA sequences set forth in Table I.

TABLE I

| siRNA sequence | DNA Target Sequence | Target mRNA Sequence(s) |
|---|---|---|
| AGA AUU UGA CAA GGC | AGC CGT GCC TTG TCA | 1) NM_001455.3 |
| ACG UCG | AAT TCT | (SEQ ID NO: 7) (human); |
| (SEQ ID NO: 1) | (SEQ ID NO: 12) | 2) NM_201559.2 |
| | | (SEQ ID NO: 8) (human); |
| | | and |
| | | 3) NM_019740.2 |
| | | (SEQ ID NO: 10) (mouse) |
| AAA CAC GGU ACU GUU | TCC TTC AAC AGT ACC | NM_019740.2 |
| GAA GGA | GTG TTT | (SEQ ID NO: 10) (mouse) |
| (SEQ ID NO: 2) | (SEQ ID NO: 13) | |
| AUU UCC UUG GUU GCC | GCT CTG GGC AAC CAA | NM_019740.2 |
| CAG AGC | GGA AAT | (SEQ ID NO: 10) (mouse) |
| (SEQ ID NO: 3) | (SEQ ID NO: 14) | |
| AUA GUC UGC AUG GGU | TCA GTC ACC CAT GCA | NM_019740.2 |
| GAC UGA | GAC TAT | (SEQ ID NO: 10) (mouse) |
| (SEQ ID NO: 4) | (SEQ ID NO: 15) | |
| AUG UUG CUG ACA GAA | GTC GAA TTC TGT CAG | NM_001455.3 |
| UUC GAC | CAA CAT | (SEQ ID NO: 7) (human) |
| (SEQ ID NO: 44) | (SEQ ID NO: 48) | |
| AUG AAU CGA CUA UGC | GTC ACT GCA TAG TCG | NM_001455.3 |
| AGU GAC | ATT CAT | (SEQ ID NO: 7) (human) |
| (SEQ ID NO: 45) | (SEQ ID NO: 49) | |
| AUG UUA UCC AGC AGG | GGA CGA CCT GCT GGA | NM_001455.3 |
| UCG UCC | TAA CAT | (SEQ ID NO: 7) (human) |
| (SEQ ID NO: 46) | (SEQ ID NO: 50) | |
| AUA GUG UGA CAU GGA | TTC TCT TCC ATG TCA | NM_001455.3 |
| AGA GAA | CAC TAT | (SEQ ID NO: 7) (human) |
| (SEQ ID NO: 47) | (SEQ ID NO: 51) | |

In an embodiment, the siRNA has at least about 70% or more, e.g., about 80% or more, about 90% or more, about 91% or more, about 92% or more, about 93% or more, about 94% or more, about 95% or more, about 96% or more, about 97% or more, about 98% or more, or about 99% or more, identity to any one or more of SEQ ID NOs: 1-4 and 44-47.

The method comprises inhibiting the activity of the foxo3a gene or gene product in dendritic cells in the mammal. The foxo3a siRNA may inhibit the activity of the foxo3a gene or gene product in any suitable manner. The foxo3a gene product may include, for example, foxo3a mRNA or FOXO3A protein. In an embodiment, the activity of the foxo3a gene is inhibited by decreasing endogenous expression of the foxo3a gene. The foxo3a siRNA may inhibit or downregulate to some degree the expression of the protein encoded by a foxo3a gene, e.g., at the DNA, RNA, or other level of regulation. In this regard, a dendritic cell comprising a foxo3a siRNA expresses no or lower levels of foxo3a mRNA or FOXO3A protein as compared to a dendritic cell that lacks a foxo3a siRNA.

Without being bound to a particular theory, it is believed that inhibiting the activity of the foxo3a gene or gene product in dendritic cells increases the ability of the dendritic cell to stimulate an antigen-specific immune response as compared to a dendritic cell in which the activity of the foxo3a gene or gene product is not inhibited. It is further believed that inhibiting the activity of the foxo3a gene or gene product in dendritic cells reduces the tolerogenicity of the dendritic cell as compared to a dendritic cell in which the activity of the foxo3a gene or gene product is not inhibited. In this regard, inhibiting the activity of the foxo3a gene or gene product in dendritic cells may produce dendritic cells having a phenotype that is associated with reduced tolerogenicity and/or increased T cell stimulatory capacity. For example, inhibiting the activity of the foxo3a gene or gene product in the dendritic cells may increase dendritic cell CD80 and/or interleukin (IL)-6 expression and/or may decrease dendritic cell arginase, transforming growth factor (TGF)-β, and/or indolamine-2-3-deoxygenase (IDO) expression.

In an embodiment of the invention, the method may also comprise administering a cancer antigen to a mammal. The cancer antigen can be any cancer antigen. Cancer antigens are molecules (e.g., polypeptide, lipid, carbohydrate, etc.) that are uniquely expressed by tumor cells, or significantly over-expressed by tumor cells as compared to non-tumor cells, such that an immune response to the antigen results in the more rapid destruction of tumor cells as compared to normal (non-cancerous) cells. The cancer antigen administered according to the inventive methods may be a protein, polypeptide, and/or nucleic acid encoding the cancer antigen.

The cancer antigen can be an antigen expressed by any cell of any cancer or tumor. For example, the cancer antigen can be an antigen expressed by any cell of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, uterine cancer, esophageal cancer, cervical cancer, gastrointestinal carcinoid tumor, lymphoid and other hematopoietic tumors, Hodgkin lymphoma, B cell lymphoma, bronchial squamous cell cancer, hypopharynx cancer, kidney cancer, larynx cancer, liver cancer, pancreatic cancer, carcinoma, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. Preferably, the cancer antigen is a prostate cancer antigen, a renal cancer antigen, or a melanoma antigen.

More specific examples of cancer antigens include polypeptides such as early prostate cancer antigen-2 (EPCA-2), Ig-idiotype of B cell lymphoma, thyroid medullary, small cell lung cancer, colon and/or bronchial squamous cell cancer, BAGE of bladder, melanoma, breast, and squamous-cell carcinoma, gp75 of melanoma, oncofetal antigen of melanoma; carbohydrate/lipids such as muci mucin of breast, pancreas, and ovarian cancer, GM2 and GD2 gangliosides of melanoma; oncogenes such as mutant p53 of carcinoma, mutant ras of colon cancer and HER21neu proto-onco-gene of breast carcinoma; and viral products such as human papilloma virus polypeptides of squamous cell cancers of cervix and esophagus. Additional examples of cancer antigens, including the nucleotide sequences that encode them and the amino acid sequences that contain them, are identified in Table II. Accordingly, the cancer antigen can comprise, consist, or consist essentially of any of SEQ ID NOs: 16-29. A preferred cancer antigen is the prostate cancer antigen prostatic acid phosphatase (PAP).

TABLE II

| | Nucleotide | Amino Acid |
|---|---|---|
| Antigen | Sequence | Sequence |
| Prostate Cancer Antigens | | |
| prostate-specific antigen (PSA) | SEQ ID NO: 16 | SEQ ID NO: 23 |
| (KLK3) | | |
| prostate-specific membrane antigen | SEQ ID NO: 17 | SEQ ID NO: 24 |
| (PSMA) (FOLH1) | | |
| Prostate Stem Cell Antigen | SEQ ID NO: 18 | SEQ ID NO: 25 |
| (PSCA) | | |
| Prostatic acid phosphatase (PAP) | SEQ ID NO: 19 | SEQ ID NO: 26 |
| (ACPP) | | |
| telomerase reverse transcriptase | SEQ ID NO: 20 | SEQ ID NO: 27 |
| (TERT) | | |
| survivin (BIRC5) | SEQ ID NO: 21 | SEQ ID NO: 28 |
| mucin-1 (MUC1) (L-BLP25) | SEQ ID NO: 22 | SEQ ID NO: 29 |
| Melanoma Antigens | | |
| gp100 | SEQ ID NO: 52 | SEQ ID NO: 53 |
| MART-1 | SEQ ID NO: 54 | SEQ ID NO: 55 |
| p15 | SEQ ID NO: 56 | SEQ ID NO: 57 |
| mutant cyclin-dependent kinase 4 | SEQ ID NO: 58 | SEQ ID NO: 59 |
| (CDK4) | | |
| NY-ESO-1 | SEQ ID NO: 60 | SEQ ID NO: 61 |
| MAGE 1 | SEQ ID NO: 62 | SEQ ID NO: 63 |
| MAGE 2 | SEQ ID NO: 64 | SEQ ID NO: 65 |
| MAGE 3 | SEQ ID NO: 66 | SEQ ID NO: 67 |
| mesothelin | SEQ ID NO: 68 | SEQ ID NO: 69 |
| tyrosinase tumor antigen | SEQ ID NO: 70 | SEQ ID NO: 71 |
| tyrosinase related protein (TRP)-1 | SEQ ID NO: 72 | SEQ ID NO: 73 |
| TRP-2 | SEQ ID NO: 74 | SEQ ID NO: 75 |
| Renal Tumor Antigens | | |
| Carbonic Anhydrase IX | SEQ ID NO: 76 | SEQ ID NO: 77 |

The inventive method may comprise administering the cancer antigen and the foxo3a siRNA to the mammal in any suitable sequence. In an embodiment, the method comprises administering the cancer antigen to the mammal before administering the foxo3a siRNA to the mammal or administering the foxo3a siRNA to the mammal after administering the cancer antigen to the mammal. In another embodiment, the method comprises administering the foxo3a siRNA to the mammal before administering the cancer antigen to the mammal or administering the cancer antigen to the mammal after administering the foxo3a siRNA to the mammal. In still another embodiment, the method comprises administering the cancer antigen and the foxo3a siRNA to the mammal simultaneously. Alternatively, the method may comprise administering the cancer antigen and the foxo3a siRNA to the mammal in a combination of any of the sequences described herein. Accordingly, the activity of the foxo3a gene or gene product may be inhibited before, during, after, or a combination thereof, the administration of the cancer antigen.

The cancer antigen and the foxo3a siRNA may be administered to the mammal in any suitable manner. In an embodiment, the method comprises administering the cancer antigen and/or the foxo3a siRNA directly to the mammal. In this regard, the method may comprise administering the cancer antigen and/or the foxo3a siRNA directly into a tumor.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments of the method, the nucleic acid does not comprise any insertions, deletions, inversions, and/or substitutions. However, it may be suitable in some instances for the nucleic acid to comprise one or more insertions, deletions, inversions, and/or substitutions.

The nucleic acids may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., Molecular Cloning: A Laboratory Manual, 3^{rd} ed., Cold Spring Harbor Press, Cold Spring Harbor, N.Y., 2001. The nucleic acids can be constructed based on chemical synthesis and/or enzymatic ligation reactions using procedures known in the art. See, for example, Sambrook et al., supra, and Ausubel et al., Current Protocols in Molecular Biology, Greene Publishing Associates and John Wiley & Sons, NY, 1994. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxy aminomethyl-2-thiouracil, beta-D-mannosylqueosine, 5′-methoxyearboxymethyluracil, 5-methoxyuracil, 2-methylthio-N⁶-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can be incorporated into a recombinant expression vector. In this regard, an embodiment of the invention uses recombinant expression vectors comprising any of the nucleic acids described herein. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole. However, parts of the vectors can be naturally-occurring. The recombinant expression vectors can comprise any type of nucleotides, including, but not limited to DNA and RNA, which can be single-stranded or double-stranded, which can be synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vectors can be any suitable recombinant expression vector. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and XNM1149, also can be used. Examples of plant expression vectors include pBI01, pBI101.2, pBI101.3, pBI121, and pBIN19 (Clontech). Examples of animal expression vectors include pEUK-Cl, pMAM, and pMAMneo (Clontech). The recombinant expression vector may be a viral vector, e.g., a retroviral vector.

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, SV40, bovine papilloma virus, and the like.

The recombinant expression vector may comprise regulatory sequences, such as transcription and translation initiation and termination codons, which are specific to the type of host (e.g., bacterium, fungus, plant, or animal) into which the vector is to be introduced, as appropriate, and taking into consideration whether the vector is DNA- or RNA-based.

The recombinant expression vector can include one or more marker genes, which allow for selection of transduced hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter operably linked to the nucleotide sequence encoding the cancer antigen, the foxo3a siRNA, or foxo3a (described in more detail below). The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

The recombinant expression vectors described herein may be used, for example, to transduce a dendritic cell (DC). The dendritic cell may be a mouse dendritic cell or a human dendritic cell. Preferably, the dendritic cell is a human dendritic cell. For purposes herein, the dendritic cell can be any dendritic cell, such as a cultured dendritic cell or a dendritic cell obtained from a mammal. If obtained from a mammal, the dendritic cell can be obtained from numerous sources, including but not limited to tumor biopsy or necropsy, blood, bone marrow, lymph node, or other tissues or fluids. Dendritic cells can also be enriched for or purified. The dendritic cell may be a dendritic cell isolated from a human. The dendritic cell can be any type of dendritic cell. For example, the dendritic cell may be a conventional dendritic cell (cDC), a plasmacytoid dendritic cell (pDC), a IDO+/CD8α+ DC, or a dendritic cell having the phenotype described in Example 1. In a preferred embodiment, the dendritic cell is a tumor-associated dendritic cell (TADC). In a particularly preferred embodiment, the dendritic cell is a melanoma TADC or a prostate cancer TADC.

Another embodiment of the method of the invention comprises (a) obtaining dendritic cells from the mammal; (b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells; (c) inhibiting the activity of the foxo3a gene or gene product in the dendritic cells by delivering an siRNA selected from the group consisting of (a) SEQ ID NOs: 1-4 and 44-47 and (b) siRNAs having at least 95% identity to any one of SEQ ID NOs: 1-4 and 44-47 and a nucleotide length of about 18 to about 30 to the dendritic cells to produce dendritic cells having decreased foxo3a gene or gene product activity; and (d) administering the antigen-expressing dendritic cells having decreased foxo3a gene or gene product activity to the mammal, thereby enhancing an immune response to the cancer antigen in the mammal. The detailed discussions of aspects of other embodiments are applicable to similar aspects of this embodiment, e.g., the description of suitable siRNA.

The method comprises obtaining dendritic cells from the mammal. The dendritic cells can be obtained from the mammal by any suitable means known in the art. For example, the dendritic cells can be obtained from the mammal by, for example, a blood draw, leukapheresis, bone marrow biopsy, and/or tumor biopsy or necropsy. The dendritic cells can be obtained from any suitable source, including any of the sources described herein with respect to other aspects of the invention.

The method comprises causing the dendritic cells to express the cancer antigen. Antigen-expressing dendritic cells present the antigen on the surface of the dendritic cell so that the antigen may be recognized by T cells. In an embodiment, dendritic cells may be caused to express the cancer antigen by exposing the dendritic cells to the cancer antigen in culture under conditions promoting uptake and processing of the antigen. For example, dendritic cells may be pulsed with a cancer antigen. Methods of exposing the dendritic cells to the cancer antigen to promote uptake and processing of the antigen are generally known in the art (see, e.g., Paczesny et al., J. Exp. Med., 199(11): 1503-11 (2004)). In a preferred embodiment, causing the dendritic cells to express the cancer antigen further comprises exposing the dendritic cells to granulocyte macrophage-colony stimulating factor (GM-CSF).

In another embodiment, dendritic cells may be caused to express the cancer antigen by transducing the dendritic cells with a nucleic acid sequence encoding the cancer antigen to produce antigen-expressing dendritic cells. A number of transduction techniques are generally known in the art (see, e.g., Graham et al., Virology, 52: 456-467 (1973); Sambrook et al., supra; Davis et al., Basic Methods in Molecular Biology, Elsevier (1986); and Chu et al., Gene, 13: 97 (1981)). Transduction methods include calcium phosphate co-precipitation (see, e.g., Graham et al., supra), direct microinjection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Felgner et al., Proc. Natl. Acad. Sci. USA, 84: 7413-7417 (1987)), and nucleic acid delivery using high velocity microprojectiles (see, e.g., Klein et al., Nature, 327: 70-73 (1987)). The cancer antigen may be any of the cancer antigens described herein.

The method comprises inhibiting the activity of the foxo3a gene or gene product in the dendritic cells by delivering an siRNA selected from the group consisting of (a) SEQ ID NOs: 1-4 and 44-47 and (b) an siRNA having at least 95% identity to any one of SEQ ID NOs: 1-4 and 44-47 and a nucleotide length of about 18 to about 30 to the dendritic cells to produce dendritic cells having decreased foxo3a gene or gene product activity. An siRNA may be delivered to the dendritic cells in any suitable manner. For example, an siRNA may be delivered to the dendritic cells by culturing the dendritic cells with an siRNA, which may comprise physically contacting the dendritic cells with the siRNA to facilitate uptake of the siRNA by the dendritic cell so that the siRNA binds to a target foxo3a nucleotide sequence in the cell for a time and under conditions sufficient to decrease expression of foxo3a. Alternatively, the siRNA may be delivered to the dendritic cell by microinjection of the siRNA directly into the dendritic cell or by transducing the dendritic cell with a DNA sequence encoding the siRNA. In an embodiment of the invention, inhibiting the activity of the foxo3a gene or gene product in the dendritic cells may include combining the siRNA with sipuleucel-T (e.g., PROVENGE™ sipuleucel-T, Dendreon Corporation, Seattle, Wash.).

The method also comprises administering the antigen-expressing dendritic cells having decreased foxo3a gene or gene product activity to the mammal. In a preferred embodiment, the method comprises administering the antigen-expressing dendritic cells having decreased foxo3a gene or gene product activity directly into a tumor. The dendritic cells may be administered to the mammal in any suitable manner, and may include those methods described herein with respect to other aspects of the invention.

Another embodiment of the invention comprises (a) obtaining T cells and dendritic cells from the mammal; (b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells; (c) inhibiting the activity of the foxo3a gene or gene product in the dendritic cells by delivering an siRNA selected from the group consisting of (a) SEQ ID NOs: 1-4 and 44-47 and (b) siRNAs having at least 95% identity to any one of SEQ ID NOs: 1-4 and 44-47 and a nucleotide length of about 18 to about 30 to the dendritic cells to produce dendritic cells having decreased foxo3a gene or gene product activity; (d) culturing the antigen-expressing dendritic cells having decreased foxo3a gene or gene product activity with the T cells; and (e) administering the T cells to the mammal, thereby enhancing an immune response to the cancer antigen in the mammal. The detailed discussions of aspects of other embodiments are applicable to similar aspects of this embodiment, e.g., the description of suitable siRNA.

The method comprises obtaining T cells from the mammal. The T cells can be obtained from the mammal by any suitable means known in the art. For example, the T cells can be obtained from the mammal by a blood draw or leukapheresis. The T cells may, optionally, be modified to express a T cell receptor specific for the cancer antigen.

For purposes herein, the T cell can be any T cell, such as a cultured T cell, e.g., a primary T cell, or a T cell from a cultured T cell line or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell or a combination thereof, as described in Shafer-Weaver et al., Cancer Res., 69(15): 6256-64 (2009)).

The method comprises causing the dendritic cells to express the cancer antigen. The dendritic cells may be caused to express the cancer antigen by any of the methods described herein. The cancer antigen may be any of the cancer antigens described herein.

The method comprises inhibiting the activity of the foxo3a gene or gene product in the dendritic cells by delivering an siRNA selected from the group consisting of (a) SEQ ID NOs: 1-4 and 44-47 and (b) an siRNA having at least 95% identity to any one of SEQ ID NOs: 1-4 and 44-47 and a nucleotide length of about 18 to about 30 to the dendritic cells to produce dendritic cells having decreased foxo3a gene or gene product activity. An siRNA may be delivered to the dendritic cells in any suitable manner, for example, using any of the methods described herein.

The method comprises exposing the antigen-expressing dendritic cells having decreased foxo3a gene or gene product activity to the T cells. The dendritic cells may be exposed to the T cells in any suitable manner. For example, the dendritic cells having decreased foxo3a gene or gene product activity may be cultured with the T cells for a time and under conditions sufficient to stimulate the T cells to immunologically recognize the cancer antigen. To stimulate the T cells, the dendritic cells may directly physically contact the T cells.

The method also comprises administering the T cells to the mammal. In a preferred embodiment, the method comprises administering the T cells directly into a tumor. The T cells may be administered to the mammal in any suitable manner, and may include those methods described herein with respect to other aspects of the invention. For example, the method may include systemically adminstering T cells to the mammal.

The inventive methods comprise enhancing an immune response to a cancer antigen in a mammal. An antigen specific-immune response can be characterized by the production of lymphocytes that are capable of recognizing and differentiating the antigen from other antigens and mediating the destruction of the antigen-bearing cell. An antigen-specific immune response also can be characterized by the production, maturation, activation, and/or recruitment of antigen presenting cells, and/or the expression of cytokines in response to stimulation by the antigen.

An antigen-specific immune response is enhanced in accordance with the invention if the immune response to a particular antigen is greater, quantitatively or qualitatively, after administration of a foxo3a siRNA, dendritic cells, or T cells described above as compared to the immune response in the absence of the administration of foxo3a siRNA, dendritic cells, or T cells described above. A quantitative increase in an immune response encompasses an increase in the magnitude or degree of the response. The magnitude or degree of an immune response can be measured on the basis of any number of known parameters, such as an increase in the level of antigen-specific cytokine production (cytokine concentration), an increase in the number of lymphocytes activated (e.g., proliferation of antigen-specific lymphocytes) or recruited, and/or an increase in the production of antigen-specific antibodies (antibody concentration), etc. A qualitative increase in an immune response encompasses any change in the nature of the immune response that renders it more effective at combating a cancer antigen or the cancer disease itself. Methods of measuring the immune response are known in the art. For example, measuring the types and levels of cytokines produced can measure the immune response. An enhanced immune response may be characterized by an increase in the production of cytokines such as any one or more of IFN-γ, TNFα, and granzyme B, and/or stimulating a cell-mediated immune response, such as the proliferation and activation of T-cells and/or macrophages specific for the antigen. Alternatively or additionally, an enhanced immune response may be characterized by a decrease in the production of anti-inflammatory and/or suppressive mediators such as any one or more of TGF-beta, interleukin (IL)-10, and vascular endothelial growth factor (VEGF), and/or a decrease in the number and/or frequency of FOXP3⁺ T cells. In a preferred embodiment, an enhanced immune response is characterized by any one or more of an increase in T cell stimulation, an increase in T cell proliferation, and an increase in T cell IFNγ and/or granzyme B secretion. “T cell stimulation” as used herein refers to the elicitation of the signal transduction pathways characteristic of an immune response, which signal transduction pathways are initiated by the binding of the T cell receptor (TCR) with the appropriate antigen-MHC complex. Methods of determining whether a T cell is stimulated by an antigen, e.g., the contacting antigen, are known in the art and include, for example, cytokine release assays, e.g., ELISA assays, ELISpot assays, and qPCR assays, cytotoxicity assays, and proliferation assays, and the like. Qualitative and quantitative enhancements in an immune response can occur simultaneously, and are not mutually exclusive.

The cancer antigen, foxo3a siRNA, antigen-expressing dendritic cells having decreased foxo3a gene or gene product activity, and T cells that have been exposed to the dendritic cells having decreased foxo3a gene or gene product activity, all of which are collectively referred to as “immune enhancing materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) protein preparation is one in which the protein is more pure than the protein in its natural environment within a cell. Such proteins may be produced, for example, by standard purification techniques, or by recombinant expression. In some embodiments, a preparation of a protein is purified such that the protein represents at least about 50%, e.g., at least about 60%, at least about 70%, at least about 80%, at least about 90%, or about 100%, of the total protein content of the preparation.

For administration to a mammal, the immune enhancing materials may be formulated into a composition, such as a pharmaceutical composition. In this regard, the method comprises administering a pharmaceutical composition comprising any one or more of the immune enhancing materials and a pharmaceutically acceptable carrier. The pharmaceutical compositions may contain any one or more of the immune enhancing materials. Alternatively, the pharmaceutical composition can comprise any one or more of the immune enhancing materials in combination with other pharmaceutically active agents or drugs.

One or more of the immune enhancing materials can be provided in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular immune enhancing material, as well as by the particular method used to administer the immune enhancing material. Accordingly, there are a variety of suitable formulations of the pharmaceutical composition. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The concentration of the immune enhancing material in the pharmaceutical formulations can vary, e.g., from less than about 1%, usually at least about 10%, to as much as about 20% or even to about 50% or more by weight, and can be selected primarily by fluid volumes, and viscosities, in accordance with the particular mode of administration selected.

Methods for preparing administrable (e.g., parenterally administrable) compositions are known or apparent to those skilled in the art and are described in more detail in, for example, Remington: The Science and Practice of Pharmacy, Lippincott Williams & Wilkins; 21st ed. (May 1, 2005).

The following formulations for parenteral (e.g., subcutaneous, intravenous, intraarterial, intramuscular, intradermal, interperitoneal, and intrathecal) administration are merely exemplary and are in no way limiting. More than one route can be used to administer the immune enhancing material, and in certain instances, a particular route can provide a more immediate and more effective response than another route.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The immune enhancing materials can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the immune enhancing materials in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose scaled containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The requirements for effective pharmaceutical carriers for parenteral compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

For purposes of the invention, the amount or dose of the immune enhancing material administered should be sufficient to effect the desired biological response, e.g., a therapeutic or prophylactic response, in the subject or animal over a reasonable time frame. The dose will be determined by the efficacy of the particular immune enhancing material and the condition of the mammal (e.g., human), as well as the body weight of the mammal (e.g., human) to be treated. The dose of the immune enhancing material also will be determined by the existence, nature and extent of any adverse side effects that might accompany the administration of a particular immune enhancing material. Typically, the attending physician will decide the dosage of the immune enhancing material with which to treat each individual patient, taking into consideration a variety of factors, such as age, body weight, general health, diet, sex, immune enhancing material to be administered, route of administration, and the severity of the condition being treated. It will be appreciated by one of skill in the art that various diseases or disorders could require prolonged treatment involving multiple administrations, perhaps using the immune enhancing material in each or various rounds of administration. By way of example and not intending to limit the invention, the dose of the immune enhancing material can be about 0.001 to about 1000 mg/kg body weight of the subject being treated/day, from about 0.01 to about 10 mg/kg body weight/day, about 0.01 mg to about 1 mg/kg body weight/day.

For purposes of the invention, an assay, which comprises comparing the extent to which target cells are lysed or IFN-γ is secreted by T cells upon administration of a particular dose of the immune enhancing materials to a mammal, among a set of mammals of which is each given a different dose of the immune enhancing materials, could be used to determine a starting dose to be administered to a mammal. The extent to which target cells are lysed or IFN-γ is secreted upon administration of a certain dose can be assayed by methods known in the art.

When the immune enhancing materials are administered with one or more additional therapeutic agents, one or more additional therapeutic agents can be coadministered to the mammal. By “coadministering” is meant administering one or more additional therapeutic agents and the immune enhancing materials sufficiently close in time such that the immune enhancing materials can enhance the effect of one or more additional therapeutic agents. In this regard, the immune enhancing material can be administered first and the one or more additional therapeutic agents can be administered second, or vice versa. Alternatively, the immune enhancing material and the one or more additional therapeutic agents can be administered simultaneously. Exemplary therapeutic agents that can be co-administered with the immune enhancing material include IL-2, anti-cytotoxic T lymphocyte antigen (CTLA)-4 antibodies, or anti-programmed death (PD)-1 antibodies. It is believed that IL-2 enhances the therapeutic effect of the immune enhancing material. For purposes of the inventive methods, wherein dendritic cells or T cells are administered to the mammal, the cells can be cells that are allogeneic or autologous to the mammal.

As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

An embodiment of the invention comprises a method of treating or preventing cancer by enhancing an immune response to a cancer antigen in a mammal according to any of the methods described herein. The cancer may be any cancer, for example, any of the cancers described herein with respect to the cancer antigen. Preferably, the cancer is prostate cancer, renal cancer, or melanoma.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

The invention also provides a method of suppressing an immune response to an autoimmune disease antigen in a mammal. The method comprises administering the autoimmune disease antigen to the mammal and increasing the activity of the foxo3a gene or gene product in dendritic cells in the mammal by administering a nucleic acid encoding foxo3a selected from the group consisting of (i) SEQ ID NOs: 5-6 and (ii) sequences having at least 95% identity to SEQ ID NO: 5 or 6 to the mammal, thereby suppressing an immune response to the autoimmune disease antigen in the mammal.

In an embodiment, the method comprises administering a nucleic acid encoding foxo3a comprising SEQ ID NO: 5 (human foxo3a) or SEQ ID NO: 6 (mouse foxo3a) to the mammal. The nucleic acid encoding foxo3a can be incorporated into a recombinant expression vector as described herein with respect to other aspects of the invention. In an embodiment, the nucleic acid encoding foxo3a has at least about 70% or more, e.g., about 80% or more, about 90% or more, about 91% or more, about 92% or more, about 93% or more, about 94% or more, about 95% or more, about 96% or more or more, about 97% or more, about 98% or more, or about 99% or more, identity to SEQ ID NOs: 5 or 6.

The method comprises increasing the activity of the foxo3a gene or gene product in dendritic cells in the mammal. The a nucleic acid encoding foxo3a may increase the activity of the foxo3a gene or gene product in any suitable manner. In an embodiment, the activity of the foxo3a gene is increased by increasing endogenous expression of the foxo3a gene. The nucleic acid encoding foxo3a may increase to some degree the expression of the protein encoded by a foxo3a gene, e.g., at the DNA, RNA, or other level of regulation. In this regard, a dendritic cell comprising a nucleic acid encoding foxo3a expresses higher levels of foxo3a mRNA or FOXO3A protein as compared to a dendritic cell that has not been administered a nucleic acid encoding foxo3a.

Without being bound to a particular theory, it is believed that increasing the activity of the foxo3a gene or gene product in dendritic cells decreases the ability of the dendritic cell to stimulate an antigen-specific immune response as compared to a dendritic cell in which the activity of the foxo3a gene or gene product is not increased. It is further believed that increasing the activity of the foxo3a gene or gene product in dendritic cells increases the tolerogenicity of the dendritic cells as compared to a dendritic cell in which the activity of the foxo3a gene or gene product is not increased. In this regard, increasing the activity of the foxo3a gene or gene product in dendritic cells may produce dendritic cells having a phenotype that is associated with increased tolerogenicity and/or decreased T cell stimulatory capacity. For example, increasing the activity of the foxo3a gene or gene product in the dendritic cells may decrease dendritic cell CD80 and/or interleukin (IL)-6 expression and/or may increase dendritic cell arginase, transforming growth factor (TGF)-β, and/or indolamine-2-3-deoxygenase (IDO) expression.

The method also comprises administering an autoimmune disease antigen to the mammal. An autoimmune disease antigen, as used herein, refers to a “self” antigen or an antigen that cross reacts with a “self” antigen to which the body produces a dysfunctional immune response that causes disease. The autoimmune disease antigen can be a nucleic acid, protein, or and/or polypeptide encoding the autoimmune disease antigen. The autoimmune disease antigen can be associated with any autoimmune disease. Illustrative examples of autoimmune diseases include, but are not limited to, multiple sclerosis; psoriasis; colitis; Crohn's disease; inflammatory bowel disease; uveitis; autoimmune kidney disease; diabetic nephropathy; hepatitis; vitiligo; Addison's disease; Hashimoto's disease; Graves disease; hypoparathyroidism; Myasthenia gravis; Coombs positive hemolytic anemia; systemic lupus erthymatosis; rheumatoid arthritis, ankylosing spondylitis, Sjogren's syndrome, and Type-1 diabetes. Preferably, the autoimmune disease antigen is a multiple sclerosis antigen or a type I diabetes antigen. More specific examples of autoimmune disease antigens, including the nucleotide sequences that encode them and the amino acid sequences that contain them, are identified in Table III. In this regard, the autoimmune disease antigen can comprise, consist of, or consist essentially of any of SEQ ID NOs: 30-41.

TABLE III

| | Nucleotide | Amino Acid |
|---|---|---|
| Antigen | Sequence | Sequence |
| Multiple sclerosis antigens | | |
| myelin basic protein (MBP) | SEQ ID NO: 30 | SEQ ID NO: 36 |
| myelin proteolipid protein (PLP1) | SEQ ID NO: 31 | SEQ ID NO: 37 |
| myelin oligodendrocyte | SEQ ID NO: 32 | SEQ ID NO: 38 |
| glycoprotein (MOG) | | |
| Type I diabetes antigens | | |
| Insulin (INS) | SEQ ID NO: 33 | SEQ ID NO: 39 |
| Glutamic acid decarboxylase | SEQ ID NO: 34 | SEQ ID NO: 40 |
| (GAD1) | | |
| beta-cell zinc transporter ZnT8 | SEQ ID NO: 35 | SEQ ID NO: 41 |
| (SLC30A8) | | |

The inventive method may comprise administering the autoimmune disease antigen and the nucleic acid encoding foxo3a to the mammal in any suitable sequence. In an embodiment, the method comprises administering the autoimmune disease antigen to the mammal before administering the nucleic acid encoding foxo3a to the mammal or administering the nucleic acid encoding foxo3a to the mammal after administering the autoimmune disease antigen to the mammal. In another embodiment, the method comprises administering the nucleic acid encoding foxo3a to the mammal before administering the autoimmune disease antigen to the mammal or administering the autoimmune disease antigen to the mammal after administering the nucleic acid encoding foxo3a to the mammal. In still another embodiment, the method comprises administering the autoimmune disease antigen and the nucleic acid encoding foxo3a to the mammal simultaneously. Alternatively, the method may comprise administering the autoimmune disease antigen and the nucleic acid encoding foxo3a to the mammal in a combination of any of the sequences described herein. Accordingly, the activity of the foxo3a gene or gene product may be increased before, during, after, or a combination thereof, the administration of the autoimmune disease antigen.

The autoimmune disease antigen and the nucleic acid encoding foxo3a may be administered to the mammal in any suitable manner. In an embodiment, the method comprises administering the autoimmune disease antigen and/or the nucleic acid encoding foxo3a directly to the mammal.

An embodiment of the method of the invention comprises (a) obtaining dendritic cells from the mammal; (b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells; (c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity; and (d) administering the antigen-expressing dendritic cells having increased foxo3a gene or gene product activity to the mammal, thereby suppressing an immune response to the autoimmune disease antigen in the mammal. The detailed discussions of aspects of other embodiments are applicable to similar aspects of this embodiment, e.g., the description of suitable siRNA.

Dendritic cells may be obtained from the mammal, dendritic cells may be caused to express the autoimmune disease antigen, the nucleic acid encoding foxo3a may be delivered to the dendritic cells, and the antigen-expressing dendritic cells can be administered to the mammal in any of the ways described herein with respect to the methods of enhancing an immune response. The autoimmune disease antigen may be any of the autoimmune disease antigens described herein.

An embodiment of the invention comprises (a) obtaining T cells and dendritic cells from the mammal; (b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells; (c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity; (d) exposing the antigen-expressing dendritic cells having increased foxo3a gene or gene product activity to the T cells; and (e) administering the T cells to the mammal, thereby suppressing an immune response to the autoimmune disease antigen in the mammal. The detailed discussions of aspects of other embodiments are applicable to similar aspects of this embodiment, e.g., the description of suitable siRNA.

Dendritic cells and T cells may be obtained from the mammal, dendritic cells may be caused to express the autoimmune disease antigen, the nucleic acid encoding foxo3a may be delivered to the dendritic cells, the antigen-expressing dendritic cells can be exposed to the T cells, and the T cells can be administered to the mammal in any of the ways described herein with respect to the methods of enhancing an immune response. The autoimmune disease antigen may be any of the autoimmune disease antigens described herein.

An immune response is suppressed in accordance with the invention if the immune response is diminished, quantitatively or qualitatively, after administration of the nucleic acid encoding foxo3a, dendritic cells having increased foxo3a gene or gene product activity, or T cells that have been exposed to dendritic cells having increased foxo3a gene or gene product activity described above as compared to the immune response in the absence of the administration of a nucleic acid encoding foxo3a, dendritic cells having increased foxo3a gene or gene product activity, or T cells that have been exposed to dendritic cells having increased foxo3a gene or gene product activity. A quantitative decrease in an immune response encompasses a decrease in the magnitude or degree of the response. The magnitude or degree of an immune response can be measured on the basis of any number of known parameters, such as a decrease in the level of cytokine (e.g., antigen-specific cytokine) production (cytokine concentration), a decrease in the number of lymphocytes activated (e.g., proliferation of lymphocytes (e.g., antigen-specific lymphocytes)) or recruited, and/or a decrease in the production of antibodies (antigen-specific antibodies) (antibody concentration), etc. A qualitative decrease in an immune response encompasses any change in the nature of the immune response that renders it less effective at mediating the destruction of an autoimmune disease antigen. Methods of measuring the immune response are known in the art. For example, measuring the types and levels of cytokines produced can measure the immune response. A suppressed immune response may be characterized by a decrease in the production of cytokines such as any one or more of IFN-γ, TNF-α, and granzyme B, and/or a reduced stimulation of a cell-mediated immune response, such as a decrease in the proliferation and activation of T-cells and/or macrophages specific for the antigen. Alternatively or additionally, a suppressed immune response may be characterized by an increase in the production of any one or more of TGF-beta, IL-10, arginase, and IDO, and/or an increase in the number and/or frequency of FOXP3⁺ T cells. In a preferred embodiment, a suppressed immune response is characterized by any one or more of a decrease in T cell stimulation, an decrease in T cell proliferation, and a decrease in T cell IFNγ and/or granzyme B secretion. Qualitative and quantitative diminishment of an immune response can occur simultaneously, and are not mutually exclusive.

The nucleic acid encoding foxo3a, dendritic cells having increased foxo3a gene or gene product activity, or T cells that have been exposed to dendritic cells having increased foxo3a gene or gene product activity, all of which are collectively referred to as “immune suppressing materials” hereinafter, can be isolated and/or purified as described herein with respect to other aspects of the invention. For purposes of the invention, the amount or dose of the immune suppressing materials administered should be sufficient to effect the desired biological response, e.g., a therapeutic or prophylactic response, in the subject or animal over a reasonable time frame. The dose will be determined by the efficacy of the particular immune suppressing materials and the condition of the mammal (e.g., human), as well as the body weight of the mammal (e.g., human) to be treated. The dose of the immune suppressing materials also will be determined by the existence, nature and extent of any adverse side effects that might accompany the administration of a particular immune-suppressing materials. Typically, the attending physician will decide the dosage of the immune suppressing materials with which to treat each individual patient, taking into consideration a variety of factors, such as age, body weight, general health, diet, sex, immune suppressing materials to be administered, route of administration, and the severity of the condition being treated. By way of example and not intending to limit the invention, the dose of the immune suppressing material can be about 0.001 to about 1000 mg/kg body weight of the subject being treated/day, from about 0.01 to about 10 mg/kg body weight/day, about 0.01 mg to about 1 mg/kg body weight/day.

Carriers, formulations, and routes of administration of the nucleic acid encoding foxo3a, dendritic cells having increased foxo3a gene or gene product activity, or T cells that have been exposed to dendritic cells having increased foxo3a gene or gene product activity may be any of those described herein for the administration of the immune enhancing materials.

An embodiment of the invention comprises a method of treating or preventing an autoimmune disease, comprising suppressing an immune response to an autoimmune disease antigen in a mammal according to any of the methods described herein. The autoimmune disease may be any autoimmune disease, for example, any of the autoimmune diseases described herein with respect to the autoimmune disease antigen. Preferably, the autoimmune disease is type I diabetes or multiple sclerosis.

The following examples further illustrate the invention but, of course, should not be construed as in any way limiting its scope.

### EXAMPLES

### Experimental Mice

Transgenic adenocarcinoma of the mouse prostate (TRAMP) (Greenberg et al., PNAS, 92: 3439-3443 (1995)) and B6C3F1 nontransgenic control mice served as recipients for T cell transfer. The SV40 TAg-specific CD8⁺ TcR-I and CD4⁺ TcR-II transgenic mouse strains were bred onto a rag^{-/-} background as previously described (Geiger et al., PNAS, 89: 2985-2989 (1992)). The TcR transgenic mouse strain 37B7 bears a TcR transgene that recognizes an H-2K^{b}-restricted epitope of tyrosinase-related protein 2 (TRP-2)₁₈₀₋₁₈₈ (Singh et al., J. Immunother., 32: 129-139 (2009)). B16 tumors were injected into Foxo3a^{-/-} mice (Dejean et al., Nat. Immunol., 10: 504-513 (2009)) or C57B1/6 mice. Mice were housed under specific pathogen-free conditions and were treated in accordance with National Institutes of Health guidelines under protocols approved by the Animal Care and Use Committee of the National Cancer Institute (NCI)-Frederick Facility (Frederick, Md.).

### Human Tissues

Human tissue specimens were obtained at the time of surgical resection. Tissues were examined by the University of Maryland Surgical Pathology Department for identification of tumor and non-tumor tissues. The use of these tissues was determined to be exempt from the Federal Regulations for the Protection of Human Subjects by the NCI Office of Human Subjects Research. No specific patient information was received. TADC were analyzed from 16 patient samples. Biopsies were weighed, fixed for immunohistochemistry or digested in collagenase/DNAse for 30 min. DCs were isolated via magnetic beads conjugated PTK7 or CD304 (BDCA-4) for plasmacytoid DC (pDC) according to the manufacturer's instructions (Miltenyi Biotech Inc., Auburn, Calif.). Human peripheral blood mononuclear cells (PBMCs) were stimulated with 2 μg/ml CD8+ peptides for CMV, EBV, and Flu (“CEF” peptide pool, Mabtech, Inc., Cincinnati, Ohio). The peptides are not all restricted by HLA-A2 and react with >90% of Caucasians.

### Peptides

SV40 T Ag, TAg₅₆₀₋₅₆₈ (SEFLLEKRI) (SEQ ID NO: 42), and TRP-2₁₈₀₋₁₈₈ (SVYDFFVWL) (SEQ ID NO: 43) peptides were purchased from New England Peptide (Gardner, Mass.).

### Adoptive Transfer of Transgenic Lymphocytes

Lymph node (LN) cells from TcR-I mice were prepared in a single-cell suspension. Cell numbers were adjusted to 3×10⁶ TcR-I cells to be transferred intravenously (i.v.) into recipient mice. In some experiments, inhibitors to IDO (1-methly-D-tryptophan, 1MDT) and arginase ((S) 2-bronoethyl)-L-cysteine (BEC)) were added to the drinking water every 3 days (5 μg/ml).

### Blocking and Depletion Antibodies

A blocking anti-PD-1 antibody (Ab) (clone RMP1-14) (Yamazaki et al., J. Immunol., 175: 1586-1592 (2005)) was administered by intraperitoneal (i.p.) injection on days 0, 1, and 3 with respect to adoptive transfer of TcR-I T cells (250 μg/injection). Anti-TGF-β Ab was injected i.p. on days −1,0,1 and 2 with respect to TcR-I transfer (500 μg/injection). Anti-CD317 Ab (5 μg) (kindly provided by Dr. Marco Colonna) was injected i.p. on days −1 and 0 with respect to TcR-I transfer (Blasius et al., J. Immunol., 177: 3260-3265 (2006)).

### Cell Isolations

Prostatic tissues were removed from 14-16 week-old TRAMP mice in groups of 3-6 mice, or as indicated. Tissues were digested in a solution of collagenase and DNAse. Dendritic cells were isolated from single-cell suspensions of the prostate using the Miltenyi (Biotech Inc., Auburn, Calif.) MACS™ cell separation system and the Pan-DC magnetic beads, which consist of anti-CD11c and anti-mPDCA-1 (CD317) Ab. Cell separations were completed according to the manufacturer's instructions and consistently yielded purity of >90% CD11c⁺/CD317⁺ cells. TcR-I T cells were isolated using Thy1.1-specific antibodies and magnetic beads as described previously (Anderson et al., J. Immunol., 178: 1268-1276 (2007)).

### Flow Cytometry

Cell suspensions were blocked with Fc block, washed, and incubated with the indicated Abs purchased from BD Pharmingen (San Diego, Calif.) or eBioscience (San Diego, Calif.).

### In Vitro Proliferation Assays

Naïve TcR-I T cells were used as responder cells in a proliferation assay; 2×10⁴ T cells were stimulated with antigen and 2×10⁴ isolated prostatic DCs. After 72 h of culture, wells were pulsed with 1 μCi [³H] thymidine (Amersham, Buckinghamshire, England) for 16 h. The cells were then harvested using a Cell Harvester (Tomtec, Hamden, Conn.) and radioactivity was measured in a Liquid Scintillation Counter (WALLAC MICROBETA™ TriLux, PerkinElmer, Waltham, Mass.).

### Tolerance and Suppression Assays

Naïve TcR-I T cells were co-cultured with DC for 72 hours. TcR-I cells were then re-isolated via negative selection with magnetic beads. To assess tolerance, TcR-I T cells were subjected to secondary stimulation with normal splenocytes and TAg. After 48 hours, wells were pulsed with 1 μCi [³H] thymidine (Amersham, Buckinghamshire, England) for 12 hours. To assess suppressor activity, graded numbers of TcR-I T cells were added to 1×10⁴ responder 37B7 TRP-2-specific T cells (Singh et al., J. Immunother., 32: 129-139 (2009)), 1×10⁵ splenocytes, and 5 μM TRP-2, and incubated for 72 h at 37° C. One μCi of [³H] thymidine per well was added for an additional 16 h and harvested as described above. Previous studies have demonstrated that in this assay, tolerized T cells do not proliferate and therefore ³H-thymidine incorporation is an indication of 37B7 cell proliferation (Shafer-Weaver et al., J. Immunol., 183(8): 4848-52 (2009)).

For human assays, autologous PBMCs were cultured for 72 hours with TADC isolated from human prostate tumor tissue or irradiated PBMC. Lymphocytes were then re-isolated by negative selection using magnetic beads. To assess tolerance, cultured lymphocytes were re-stimulated with irradiated autologous PBMCs and CEF (CMV, EBV, Influenza) antigen for 48 hours. To assess suppressor activity, cultured lymphocytes were used to suppress autologous PBMC stimulation at graded suppressor:responder ratios for 48 hours. Wells were pulsed with 1 μCi [³H] thymidine (Amersham, Buckinghamshire, England) for 12 hours.

### ELISPOT Assays

Multiscreen plates (Millipore, Billerica, Mass.) were coated with 100 μl of capture Ab (R&D Systems Inc., Minneapolis, Minn.) overnight at 4° C. IFN-γ (1×10⁵) or granzyme B (2×10⁴) purified Thy 1.1⁺ TcR T cells were added to increasing concentrations of TAg₅₆₀₋₅₆₈. After incubation, plates were washed and processed as previously described (Shafer-Weaver et al., Cancer Research, 69: 6256-6264 (2009)).

### Microarray and Real Time PCR

RNA was isolated by RNAeasy Spin Columns (Qiagen, Valencia, Calif.) per the manufacturer's instructions from DCs purified from 5 TRAMP and 5 wild-type (WT) mouse prostate tissue. RNA quality was determined by Nanodrop spectrophotometer (Thermo Scientific, Wilmington, Del.). High quality RNA was sent to the Laboratory of Molecular Technologies (SAIC-Frederick, Md.) for hybridization to Mouse 1.0 ST Gene Array or Human Affymetrix Human Gene 1.0 ST array (Affymetrix, Santa Clara, Calif.). Gene expression and pathway analysis was performed with Partek (St. Louis, Mo.) and Gene Portal software. Fold changes greater than 2.1 and p<0.0001 were considered significant and verified by real-time PCR. Primers for RT-PCR were purchased from SABiosciences (Frederick, Md.) and used per the manufacturer's instructions in combination with SYBR™ Green (Applied Biosystems, Carlsbad, Calif.). Samples were run on a BioRad iCycler RT-PCR machine (Bio-Rad Laboratories, Hercules, Calif.). For each pair-wise set of samples (WT vs. TRAMP or Tumor vs. Non-Tumor) fold change was calculated by the relative expression software tool (REST Software—Corbett Research, Qiagen, Valencia, Calif.) (Pfaffl et al., Nucleic Acids Res., 30: 36 (2002)).

### Gene Silencing

DCs were isolated from TRAMP or WT prostates and cultured with 4 mixed siRNAs to foxo3u, GAPDH, or scrambled negative control siRNAs (purchased from SABiosciences (Frederick, Md.)) for 24, 48, and 72 hrs. DCs were then stained for flow cytometric analysis (CD80, CD86, CD11c), lysed for protein for western blot (FOXO3A), lysed for RNA for RT-qPCR, or added to T cell stimulation assays. Viability of DC after siRNA treatment was equivalent, irrespective of the siRNA used.

### Statistics

Statistical analyses for differences between group means were performed by unpaired Student's t test, or ANOVA. Data are presented as means±SEM or mean±S.D. as indicated. p<0.05 was considered statistically significant.

### Example 1

This example demonstrates that dendritic cells (DCs) infiltrate human prostate tumors.

Histological analyses detected strong leukocytic infiltration in biopsies of advanced prostate tumors. Flow cytometric analysis of disaggregated tumor biopsies revealed that among the CD45′ cells, 63% were CD14⁺/CD16⁺ macrophages, 21% were CD11c⁺ conventional DC (cDC), and 14% were CD123VCD304⁺/CD11c⁻ pDCs. Based on their proposed regulatory function, the pDC population was enriched using magnetic beads coupled to anti-PTK7 or anti-CD304 (Colonna et al., Nat. Immunol., 5: 1219-1226 (2004)). Enriched TADC were stained with a modified Wright-Giemsa stain after cytospin and were analyzed for pDC surface markers and co-stimulatory molecule expression. The purified cells had a plasma cell-like morphology and were CD123⁺, ILT7⁺, and CD11c⁻, consistent with human pDCs, and also expressed low levels of CD80 and CD86.

### Example 2

This example demonstrates that tumor-associated DC (TADC) have a lower stimulatory activity than autologous PBMC.

To determine the ability of human prostate TADC to stimulate T cells, enriched pDCs were cultured with autologous peripheral blood T cells and a pool of common viral antigens (cytomegalovirus (CMV), Epstein-Barr virus (EBV), and influenza virus (Flu): “CEF”), and T cell proliferation was measured. The results are shown in Table 1. Data are representative of 4 patient samples.

TABLE 1

Counts per Minute (CPM)-Patient PBMC

| [CEF] μg/ml | TADC | PBMC |
|---|---|---|
| 0 | under 250 | under 250 |
| 1 | under 250** | 4.0 × 10³ |
| 2 | 1.0 × 10³** | 7.0 × 10³ |
| 5 | 1.2 × 10³** | 6.5 × 10³ |

mean ± s.d.,
**p < 0.001 (Student's t-test)

Using this assay, it was observed that the CD123⁺ pDC from tumor biopsies (TADC) had a lower stimulatory activity than autologous PBMC (Table 1) or pDC from non-tumor tissue.

### Example 3

This example demonstrates that TADC tolerize T cells.

Given the diminished stimulatory activity shown in Example 2, the tolerogenicity of TADCs was assessed by testing their ability to tolerize peripheral blood T cells. A tolerance assay was designed wherein three days after co-culture with TADCs and CEF antigen (primary stimulation), T cells were harvested and re-stimulated with autologous PBMC and CEF antigen (secondary stimulation), and proliferation was assessed. The results are shown in Table 2. Data are representative of 2 patient samples.

TABLE 2

| | Proliferation (CPM) | |
|---|---|---|
| | TADC (source of primary | PBMC + antigen (source of |
| [CEF] μg/ml | stimulation) | primary stimulation) |
| 0 | under 250 | under 250 |
| 1 | under 250 | 1.0 × 10³ |
| 2 | 250 | 2.5 × 10³* |
| 5 | 500 | 5.0 × 10³** |

mean ± s.d.,
*p < 0.01,
**p < 0.001 (Student's t-test)

Unlike the strong proliferative response observed by T cells initially cultured with PBMC as a source of APCs, T cells initially cultured with TADCs were unable to respond to secondary stimulation by autologous PBMCs and antigen (Table 2).

### Example 4

This example demonstrates that the expression of foxo3a is upregulated in TRAMP TADCs.

To study the role of TADC in prostate cancer and the mechanisms by which they tolerize T cells, the experimental TRansgenic Adenocarcinoma of the Mouse Prostate (TRAMP) model was utilized. TRAMP mice develop autochthonous prostatic tumors due to prostate-specific expression of the SV40 T antigen (TAg). Upon entry into the TRAMP prostate, tumor-specific T cells become tolerized and acquire suppressive function (Anderson et al., J. Immunol., 178: 1268-1276 (2007); Shafer-Weaver et al., J. Immunol., 183(8): 4848-52 (2009)). Therefore, whether prostate TADC in TRAMP mice were capable of tolerizing TcR-I cells was determined.

Initially, the phenotype and gene expression profile of DCs in TRAMP was determined. The peripheral lymphoid tissues contained a small but discreet population of B220⁺ CD317⁺ DC consistent with a plasmacytoid DC phenotype. The TRAMP tumors contained a heterogeneous population of myeloid cells, the majority of which were CD11b⁺/F4/80⁺ tumor-associated macrophages (TAMs). However, the predominant population of DCs were CD11c⁺/B220⁺/BST2(CD317)⁴/CD11b⁻, which represented approximately 30% of the CD45⁺ cells in the TRAMP prostate. Interestingly, DCs with this pDC surface phenotype were also detected in the WT prostate tissue. Perfusing the prostate tumor prior to assessing phenotype or isolating DC did not change the total number of CD11c⁺/CD317⁺ cells, although there was a small decrease in CD11c⁺/F4/80⁺ cells, presumably TAMs. Additional phenotyping revealed that WT and TRAMP prostate DCs expressed low to intermediate levels of the co-stimulatory molecules CD80, CD86, and CD40 as well as MHC class II, all of which are crucial for effective priming of naïve T cells.

To obtain a more definitive understanding of gene expression by prostatic DCs, microarray analyses was used, comparing the profiles of WT and TRAMP prostate DCs. Following purification, RNA was isolated and hybridized to an Affymetrix ST 1.0 Mouse gene array. The results are shown in Table 3A. Fold change values have corresponding p<0.00001 (ANOVA). Data are representative of 4 independent microarrays for WT and TRAMP samples.

TABLE 3A

| | Fold-Change | Other Genes of | Fold-Change |
|---|---|---|---|
| Chemokines/Cytokines | TRAMP/WT | Interest | TRAMP/WT |
| cxcl10 (IP-10) | 24.1 | fasl | 2.4 |
| cxcl9 (MIG) | 6.2 | ido | 4.5 |
| ccl5 (RANTES) | 6.7 | arg | 6.2 |
| il-6 | 5.9 | inos | 3.1 |
| tgf-β | 2.4 | pdl-1 | 3.5 |
| vegf | 2.4 | stat3 | 5.6 |
| il-1-β | 2.9 | foxo3a | 4.2 |

As shown in Table 3A, a significant up-regulation of chemokine genes important for T cell chemotaxis (CXCL10, CXCL9, CCLS) was demonstrated, indicating that TADCs may actively recruit immune cells into the tumor (Anderson et al., J. Immunol., 178: 1268-1276 (2007)). Paradoxically, TRAMP DCs also over-expressed genes associated with T cell tolerance, including arginase (arg) and indolamine-2-3-deoxygenase (ido) (Table 3A) as well as several cytokines associated with prostate cancer development that can directly suppress immune cell function (tgf-β) or promote signaling pathways associated with the growth and development of prostate cancer such as il-6 and vegf (Table 3A). Furthermore, the microarray data also revealed upregulation of genes associated with signaling pathways such as jak2/stat3 and foxo3a in TADCs (Table 3A). Quantitative real-time PCR (qrt-PCR), flow cytometric analysis and ELISAs confirmed the microarray-based observations that genes associated with immune suppression (ido, arg, pd-11 and tgf-β) were upregulated in TADCs.

This example demonstrated that the expression of foxo3a and other genes associated with suppression of the immune system is increased in TADCs.

### Example 5

This example demonstrates that TADCs are poor stimulators of CD8+ T cell proliferation in vitro.

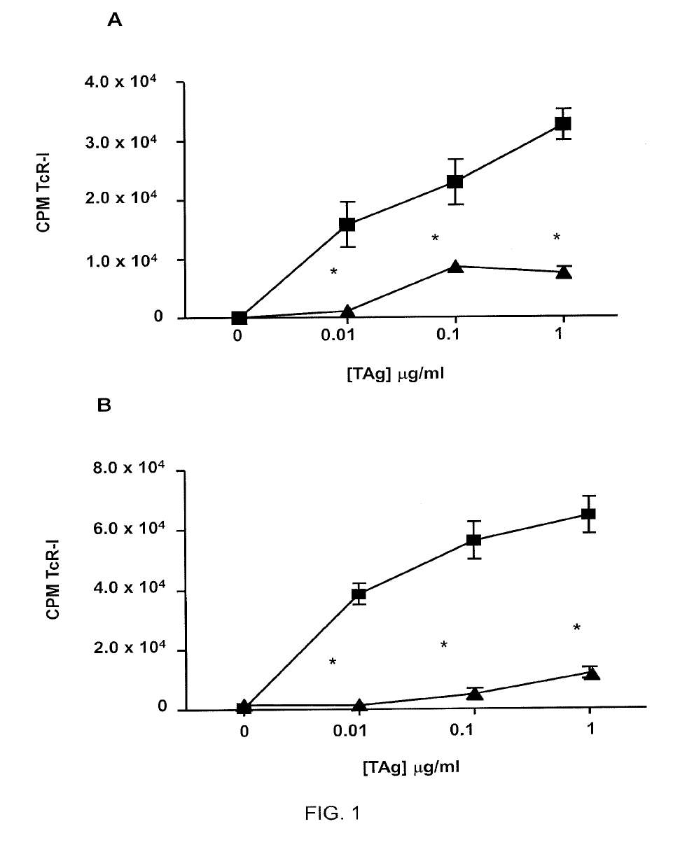

The stimulatory capacity of DCs purified from TRAMP and WT prostates was compared using naïve SV40 TAg-specific CD8⁺ (TcR-I) T cells as responder cells in the in vitro proliferation assay described above. In contrast to WT prostate DC, which stimulated TcR-I cell proliferation, TRAMP TADCs were unable to induce a strong proliferative response by TcR-I cells (FIG. 1A). These results suggest that TADCs are incapable of eliciting a T cell immune response and instead may be tolerogenic.

### Example 6

This example demonstrates that TADCs induce T cell tolerance in vitro.

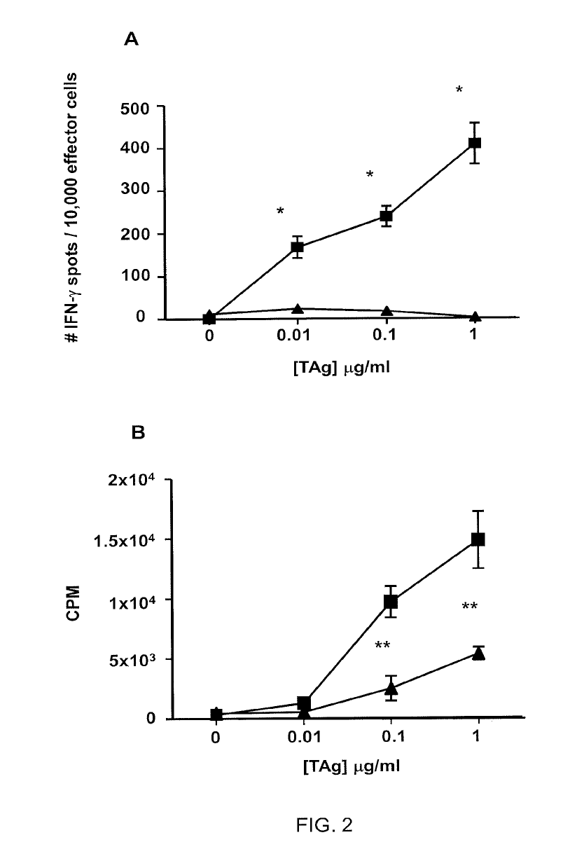

To determine whether TRAMP TADCs tolerize TcR-I T cells, prostatic DCs and naïve TcR-I T cells were co-cultured for 4 days prior to stimulating the re-isolated T cells with TAg peptide-pulsed splenic APCs. TcR-I T cells initially cultured with TRAMP TADCs did not proliferate (FIG. 1B) or produce IFN-γ (FIG. 2A) in response to secondary antigenic stimulation, whereas marked proliferative and cytokine responses were observed when WT prostate DCs were used as APCs for the primary stimulation (FIG. 1B).

Additionally, effector TcR-I T cells that were primed in vivo (Shafer-Weaver et al., J. Immunol., 183(8): 4848-52 (2009)) were co-cultured in vitro with TAg peptide and prostatic DCs from TRAMP or WT mice for 4 days prior to secondary stimulation with TAg peptide and splenic APCs, and proliferation was measured. Effector TcR-I T cells that were primed in vivo were also tolerized by TRAMP TADC (FIG. 2B).

TADC were also co-cultured in vitro with TcR-I or TcR-MeI T cells with or without antigen for 4 days prior to secondary stimulation with TAg (Table 3B) or TRP-2 (Table 3C) and splenic APCs.

TABLE 3B

| | Proliferation (CPM) for Each Source of Primary Stimulation | | | |
|---|---|---|---|---|
| TAg | | TRAMP | WT DC + | TRAMP TADC + |
| (μg/ml) | WT DC | TADC | antigen | antigen |
| 0 | 0 | 0 | 0 | 0 |
| 0.01 | 1.5 × 10⁴ | 0 | 1.5 × 10⁴ | 0 |
| 0.1 | 3.0 × 10⁴ | 0 | 2.0 × 10⁴ | <0.5 × 10⁴ |
| 1.0 | 5.0 × 10⁴ | <0.5 × 10⁴ | 3.0 × 10⁴ | <0.5 × 10⁴ |

Data representative of 2 independent trials of 3 mice per group, mean ± s.d. p < 0.0001 (Student's t-test) TRAMP vs. WT.

TABLE 3C

| | Proliferation (CPM) for Each Source of Primary Stimulation | | | |
|---|---|---|---|---|
| [TRP-2] | | TRAMP | WT DC + | TRAMP TADC + |
| (μM) | WT DC | TADC | TRP-2 | TRP-2 |
| 0 | 0 | 0 | 0 | 0 |
| 2 | 2.0 × 10⁴ | 0.8 × 10⁴ | 1.8 × 10⁴ | 0.3 × 10⁴ |
| 5 | 3.3 × 10⁴ | 2.0 × 10⁴ | 4.3 × 10⁴ | 0.5 × 10⁴ |
| 10 | 4.8 × 10⁴ | 3.0 × 10⁴ | 5.0 × 10⁴ | 0.3 × 10⁴ |

Data representative of 2 independent trials of 3 mice per group, mean ± s.d. p < 0.0001 (Student's t-test) TRAMP vs. WT.

Tolerance induction was antigen-specific, as TRAMP TADC tolerized TcR-I T cells without the addition of TAg, presumably due to antigen carry-over from the TRAMP tumor, but were unable to tolerize melanoma antigen-specific transgenic (TcR-MeI) T cells (Tables 3B and 3C). However, TRAMP TADC tolerized the TcR-MeI T cells when pulsed with the cognate melanoma antigen (Table 3C).

Taken together, these data demonstrate that DCs from the TRAMP tumor were not only ineffective at priming naïve T cells, but also tolerized naïve and effector T cells in an antigen-specific manner.

### Example 7

This example demonstrates that TADCs induce T cell suppressive activity.

Upon tumor infiltration, TcR-I cells not only become tolerized, but also acquire suppressive function (Shafer-Weaver et al., J. Immunol., 183(8): 4848-52 (2009)). Therefore, whether TADCs induce TcR-I cells to become suppressive was next determined. TcR-I cells cultured with TRAMP DCs were isolated after 4 days and used in the suppressor assay described above. The results are shown in Table 4.

TABLE 4

| | |
|---|---|
| Suppressor: | % Suppression |

| Responder | TRAMP DC | WT DC |
|---|---|---|
| 2:1 | 68* | 10 |
| 1:1 | 65* | 12 |
| 1:2 | 48* | 8 |
| 1:4 | 38* | 10 |

*p < 0.0001 WT vs. TRAMP (Student's t test).
Data are representative of 4 independent trials (3 WT and 3 TRAMP mice in each experiment), mean ± s.d.

As shown in Table 4, TcR-I T cells cultured with DC from TRAMP tumors became highly suppressive and prevented naïve T cell proliferation. In contrast, DC purified from WT prostates did not induce suppressive activity. These findings demonstrate that like TADC isolated from human prostate cancer, TRAMP TADC are highly immunosuppressive, tolerogenic, and induce suppressive activity in tumor-specific T cells.

### Example 8

This example demonstrates that depletion of TADCs results in increased TcR-I cell infiltration into the prostate.

Based on the findings that TADCs tolerized T cells and promoted suppressor cell generation in vitro, it was next determined whether depletion of TADCs in vivo enhanced T cell effector functions. TADC depletion was accomplished by injecting an anti-CD317 Ab which has been previously reported to deplete pDCs (Blasius et al., J. Immunol., 177: 3260-3265 (2006)) into TRAMP or WT mice. Anti-CD317 was injected i.p. on days −1 and 0 relative to T cell transfer. Prostate DCs were depleted in TRAMP and WT mice for up to 18 days after i.p. injection of the anti-CD317 Ab (Table 5). Only B220⁺ DC were depleted from the prostate of TRAMP mice. i.p. injection of anti-CD317 did deplete pDC (CD11c⁺/CD317⁺) but not cDC (CD11c⁺/CD317⁻) in the spleen. Prostatic tissues were harvested 6 or 12 days post T cell transfer. Prostate digests were assayed for the presence of TcR-I cells and DCs. The results are shown in Table 5.

TABLE 5

| | Day 6 | Day 12 |
|---|---|---|
| No. of Prostate DC | 1.2 × 10⁶ | 1.3 × 10⁶ |
| (control Ig) | | |
| No. of Prostate DC | 0 | 1.3 × 10⁵ |
| (anti-CD317) | | |
| No. of TcR-I cells | 4 × 10⁵ | 3 × 10⁵ |
| (control Ig) | | |
| No. of TcR-I cells | 1.6 × 10⁶ | 1.2 × 10⁶ |
| (anti-CD317) | | |

Data representative of 4 independent trials of 5 mice per group, mean ± s.d.

As shown in Table 5, significantly more TcR-I T cells were observed to infiltrate TRAMP prostates following TADC depletion, suggesting that in the absence of TADCs, TcR-I T cells underwent greater expansion and/or had increased survival.

### Example 9

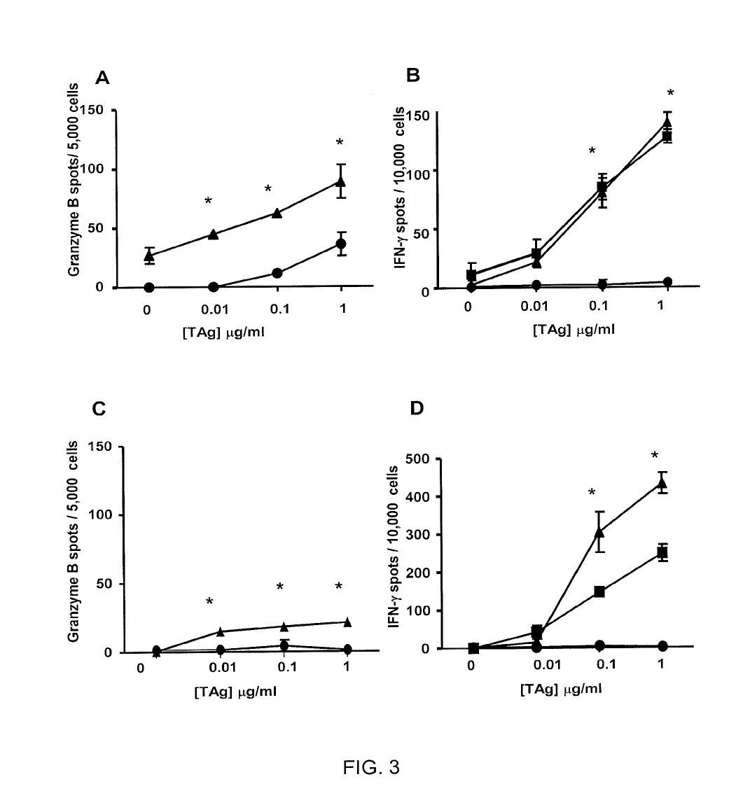

This example demonstrates that depletion of TADCs enhances cytotoxic T lymphocyte (CTL) function. It was next determined whether in vivo depletion of TADCs enhanced TcR-I effector function. TADCs were depleted via i.p. injection of anti-CD317 Ab into TRAMP mice. TcR-I T cells were then transferred into TRAMP mice with or without DC depletion. T cells were re-isolated on days 6 (FIGS. 3A and 3B) or 12 (FIGS. 3C and 3D) after transfer to assess CTL effector function.

Six days after transfer, T cells isolated from TADC-depleted TRAMP mice secreted significantly more granzyme B (FIG. 3A) and IFN-γ (FIG. 3B) compared to undepleted mice. By 12 days after transfer, granzyme B secretion (FIG. 3C) was diminished but IFN-γ secretion (FIG. 3D) was sustained at elevated levels.

T cells were also re-isolated on days 6 (Table 6) or 12 (Table 7) after transfer to assess suppressor activity.

TABLE 6

| Suppressor: | % Suppression | | |
|---|---|---|---|
| Responder | TRAMP | TRAMP + α-CD317 | WT |
| 2:1 | 100 | 25* | 5 |
| 1:1 | 90 | 40* | 10 |
| 1:2 | 72 | 38* | 11 |
| 1:4 | 70 | 30* | 10 |

Data representative of 3 independent trials with 3-5 mice per group, mean ± s.d.
*p < 0.001

TABLE 7

| | |
|---|---|
| Suppressor: | % Suppression |

| Responder | TRAMP | TRAMP + α-CD317 |
|---|---|---|
| 2:1 | 100 | 68* |
| 1:1 | 100 | 58* |
| 1:2 | 85 | 11** |
| 1:4 | 32 | 10* |

Data representative of 3 independent trials with 3-5 mice per group, mean ± s.d.
*p < 0.001;
**p < 0.0001

As shown in Tables 6 and 7, depletion of TADCs in TRAMP mice led to a significant reduction in suppressive activity by TcR-I cells 6 and 12 days after transfer (Tables 6 and 7, respectively).

This example demonstrated that the depletion of TADCs in TRAMP mice prevents the induction of T cell tolerance and reduces T cell suppressive activity.

### Example 10

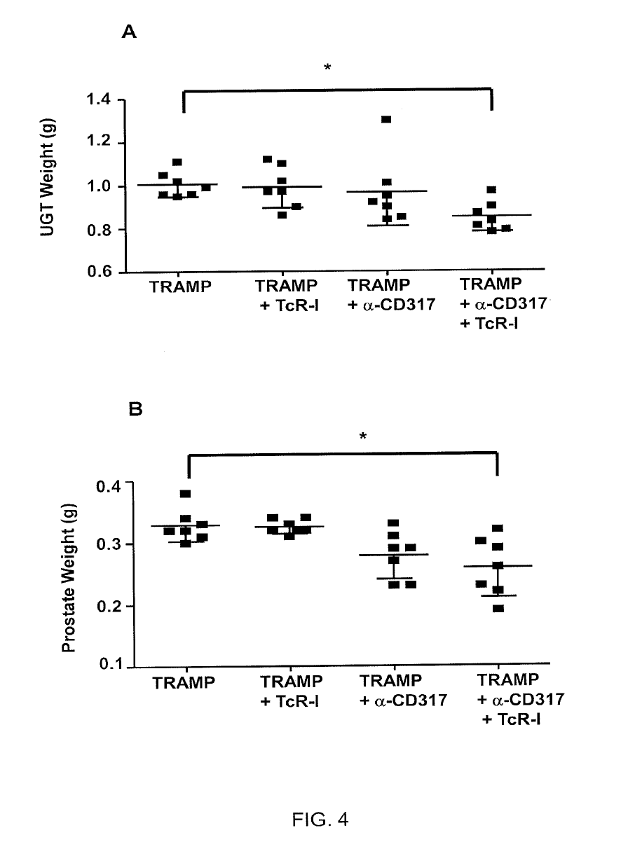

This example demonstrates that depletion of TADC results in reduced tumor size. Control antibody or anti-CD317 antibody was injected i.p. into TRAMP mice on days −1 and 0 relative to T cell transfer. Weights of total urogenital tract (UGT) and dissected prostates were obtained at day 12. The results are shown in FIG. 4A (UGT) and FIG. 4B (prostate).

Consistent with retained anti-tumor activities and diminished suppressive activity, the total UGT and prostate weights, which serve as an indicator of tumor burden in the TRAMP model, were significantly lower in TADC-depleted TRAMP mice compared to control Ig-treated mice (FIGS. 4A and 4B). Taken together, these data demonstrate that TADCs in prostate tumors were directly involved in inducing T cell tolerance and suppressive activity and are thus critical targets for ablating suppression of anti-tumor immunity.

### Example 11

This example demonstrates that blocking IDO and ARG enhances T cell activation by TRAMP DC, but does not prevent tolerization.

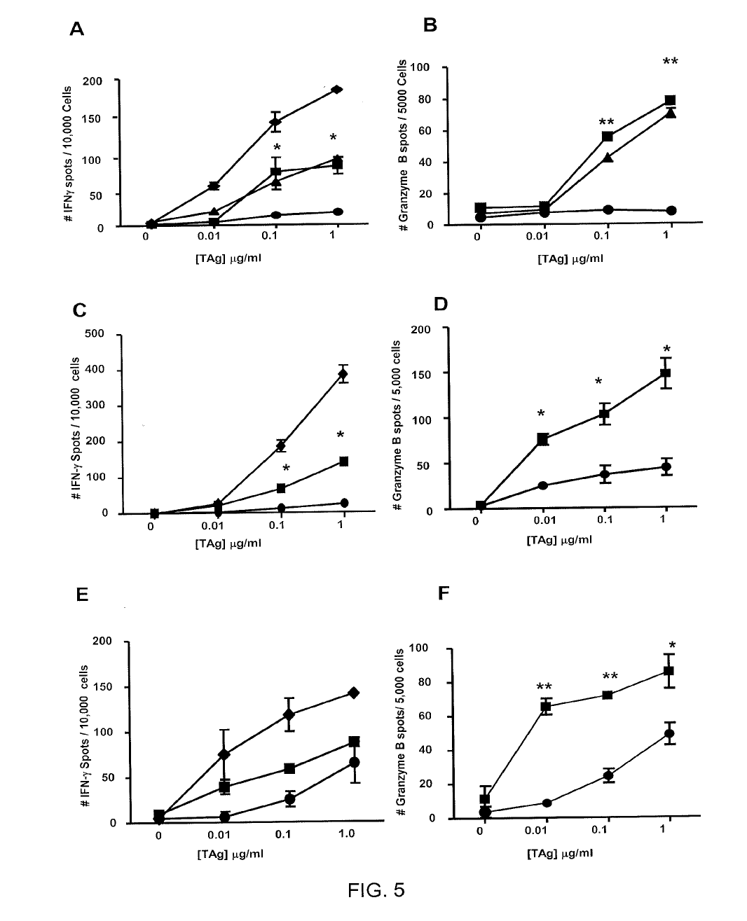

Selective amino acid catabolism is a previously-described mechanism of immune dysfunction in cancer (Bronte et al., J. Exp. Med., 201: 1257-1268 (2005); Sharma et al., J. Clin. Invest., 117: 2570-2582 (2007); Srivastava et al., Cancer Res., 70: 68-77 (2010)). The gene expression analysis described in Example 4 demonstrated that TRAMP TADCs expressed elevated levels of IDO and ARG compared to WT DCs. Therefore, the role of these catabolic enzymes was tested by supplementing the drinking water of mice with 1-methyl-D-tryptophan (1MDT) or (S)-(2-boronoethyl)-L-cysteine (BEC), inhibitors of IDO and ARG, respectively. When TRAMP mice were treated with these inhibitors prior to TcR-I T cell transfer, each significantly increased the ability of prostate-infiltrating TcR-I T cells to secrete IFN-γ (FIG. 5A) and granzyme B (FIG. 5B). Interestingly, a gradual decay in TcR-I T cell responsiveness was noted over time, thereby resulting in loss of antigen responsiveness after 12 days of treatment, and suggesting that multiple mechanisms are responsible for induction of tolerance in the TRAMP model.

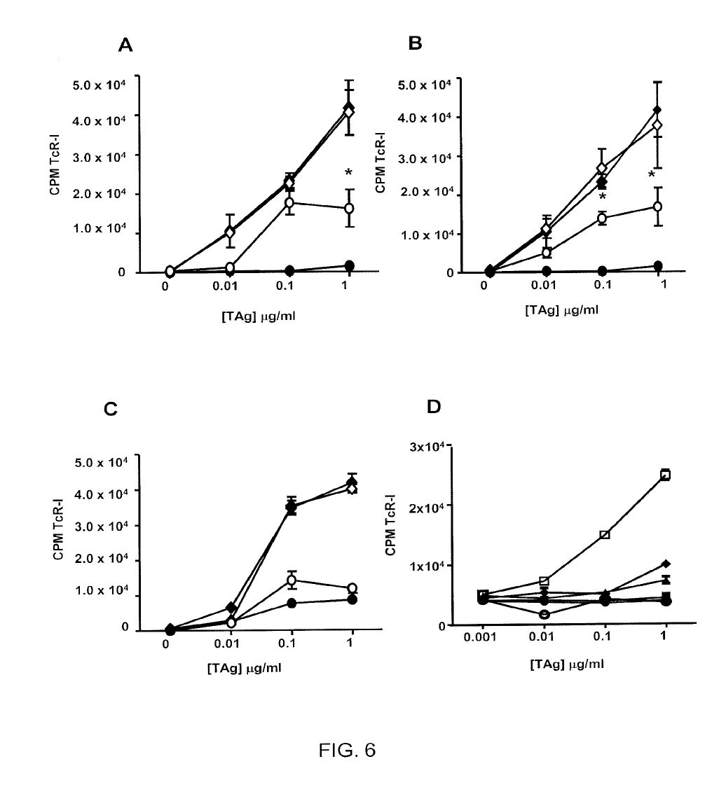

To investigate the effects of blocking IDO in vitro, 1MDT was also added to purified DC cultures to inhibit IDO activity during DC stimulation of TcR-I proliferation (FIG. 6A). Tolerance was assessed by testing secondary stimulation 4 days after primary culture with 1 MDT, a blocking agent to suppressive mediator IDO (FIG. 6D). Suppressor activity was also measured after 4 day culture with 1 MDT, and the results are shown in Table 8.

TABLE 8

| Suppressor: | % Suppression for Each Source of Primary Stimulation | | |
|---|---|---|---|
| Responder | TRAMP | TRAMP + BEC | TRAMP + 1MDT |
| 2:1 | 68 | 61 | 30 |
| 1:1 | 50 | 58 | 20 |
| 1:2 | 28 | 45 | 15 |
| 1:4 | 35 | 59 | 8 |

Blocking IDO in vitro enhanced TcR-I proliferation in response to TADCs and reduced T cell suppressor activity (FIG. 6A), but did not prevent them from becoming tolerized after longer co-culture (FIG. 6D). Due to in vitro toxicity of BEC, the ability of another inhibitor of ARG, Nor-NOHA, to block in vitro tolerization was also tested. However, blocking ARG with Nor-NOHA did not enhance TcR-I responsiveness, suggesting that the studies blocking ARG in vivo may have enhanced T cell effector functions through targets other than TADC, presumably macrophages.

This example demonstrated that blocking IDO in vitro and in vivo enhances TcR-I proliferation in response to TADCs and reduces T cell suppressor activity, but does not prevent them from becoming tolerized.

### Example 12

This example demonstrates that blocking PD-1 enhances T cell activation by TRAMP DC, but does not prevent tolerization.

To test whether PD-1 contributed to the tolerization of TcR-I T cells, anti-PD-1 was injected into TRAMP mice following T cell transfer and TcR-I T cells were isolated on days 6 and 12 after transfer to assess CTL function. Blocking PD-1 in vivo delayed the induction of T cell tolerance when tested 6 days after T cell transfer; T cells isolated from the anti-PD-1-treated group produced significantly more IFN-γ (FIG. 5C) and granzyme B (FIG. 5D) compared to isotype control Ab-treated mice. By day 12 after transfer, T cells were again observed to be hyporesponsive.

To investigate the effects of blocking PD-1 in vitro, anti-PD-1 antibody was added to cultures of TcR-I T cells and TRAMP or WT prostate DCs. Tolerance was assessed by testing secondary stimulation 4 days after primary culture with blocking agents, including anti-PD-1 antibody, to suppressive mediators (FIG. 6D). Suppressor activity was also measured after 4 day culture with blocking agents, including anti-PD-1 antibody, and the results are shown in Table 9.

TABLE 9

| Suppressor: | % Suppression for Each Source of Primary Stimulation | |
|---|---|---|
| Responder | TRAMP | TRAMP + α-PD-1 |
| 2:1 | 68 | 35 |
| 1:1 | 50 | <5 |
| 1:2 | 28 | <5 |
| 1:4 | 35 | 6 |

Blocking PD-1 in vitro increased TADC-stimulated TcR-I proliferation and reduced suppressor activity, but did not prevent tolerance during the 4 day co-culture (FIGS. 6B, 6D, and Table 9). These data suggest that blockade of PD-1/PD-L1 signaling enhanced the responsiveness and prolonged the activation of CD8⁺ T cells stimulated by TADCs, but was not sufficient to prevent tolerization.

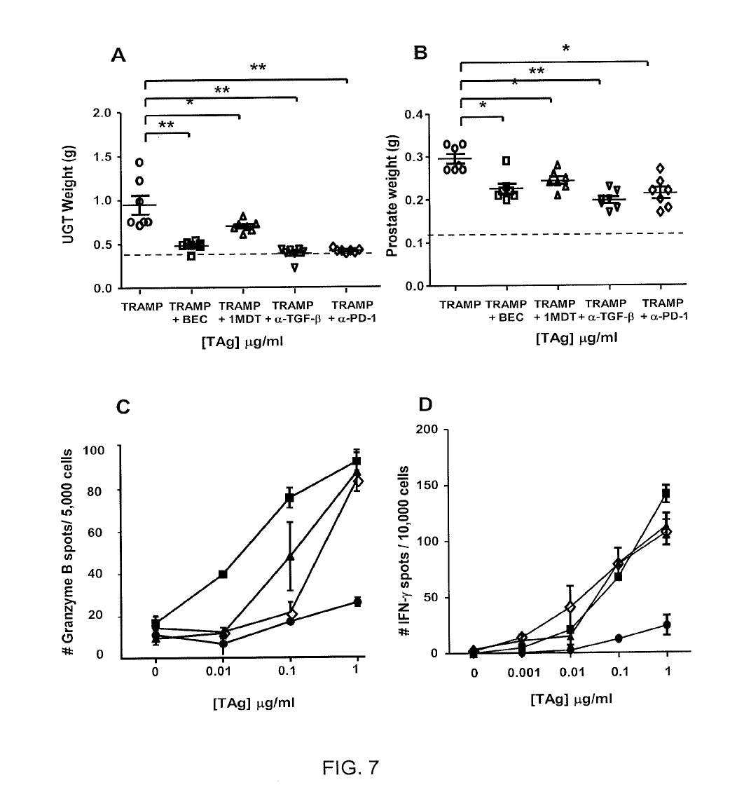

It was also tested whether PD-1 blockade in combination with blockade of the catabolic enzymes IDO or ARG enhanced T cell effector functions and prevented tolerance induction. Mice were treated with both anti-PD-1 and 1MDT and tested for granzyme (FIG. 7C) and IFN-γ secretion (FIG. 7D) on day 6 after transfer. However, no additive effect was observed.

This example demonstrated that blocking PD-1/PD-L1 signaling enhances the responsiveness and prolongs activation of CD8⁺ T cells stimulated by TADCs, but is not sufficient to prevent tolerization.

### Example 13

This example demonstrates that TGF-β is involved in the development of TcR-I suppressor cells.

TGF-β is a pleiotropic cytokine known to induce immune suppression (Li et al., Annu. Rev. Immunol., 24: 99-146 (2006)) and was up-regulated in TRAMP TADCs (Table 3, Example 4). To assess the role of TGF-(3 on the induction of T cell tolerance and suppressor cell generation in vivo, mice were treated with an anti-TGF-β antibody prior to TcR-I cell transfer. TcR-I T cells isolated from the anti-TGF-β-treated mice secreted significantly more granzyme B (FIG. 5F) than T cells from mice treated with a control antibody; surprisingly, IFN-γ expression (FIG. 5E) was not affected by TGF-β blockade.

TcR-I suppressor activity was also measured after blocking TGF-β in vivo. The results are shown in Table 10.

TABLE 10

| | % Suppression | | | |
|---|---|---|---|---|
| | TRAMP + | TRAMP + | WT + | WT + |
| Suppressor: | control | anti- | control | anti- |
| Responder | antibody | TGF-β Ab | antibody | TGF-β Ab |
| 2:1 | 78 | 42 | 22 | 22 |
| 1:1 | 37 | 30 | 17 | 20 |
| 1:2 | 25 | 15 | 12 | 10 |
| 1:4 | 15 | 7 | <5 | <5 |

Data are representative of 3 independent trials (3-5 mice per croup), mean ± s.d.,
*p < 0.05.

As shown in Table 10, TcR-I T cells from the TRAMP anti-TGF-β-treated group displayed significantly less suppressor activity than the TRAMP control antibody-treated group.

Similar to IDO and PD-1 blockade, anti-TGF-β added to in vitro cultures enhanced TADC-stimulated TcR-I cell proliferation (FIG. 6C) and blocked suppressor activity (Table 11), but did not prevent tolerance during co-culture (FIG. 6D). The exact role of TGF-β in inducing T cell tolerance will require further study.

TABLE 11

| | |
|---|---|
| Suppressor: | % Suppression |

| Responder | TRAMP | TRAMP + α-TGF-β |
|---|---|---|
| 2:1 | 68 | 7 |
| 1:1 | 50 | <5 |
| 1:2 | 28 | <5 |
| 1:4 | 35 | 5 |

This example demonstrated that TGF-β contributes to the development of TcR-I suppressor cells.

### Example 14

This example demonstrates that blocking IDO, ARG, TGF-β, or PD-1 initially delays tumor growth.

Given the enhanced effector functions seen following inhibitory molecule blockade, the effect of blocking these suppressive mediators on anti-tumor immunity was tested. TRAMP mice were untreated or treated with BEC, 1MDT, anti-TGF-β antibodies or anti-PD-1 antibodies in combination with TcR-I cell transfer. UGT weights (FIG. 7A) and prostate weights (FIG. 7B) were assessed on day 12 after TcR-I transfer.

In combination with TcR-I cell transfer, treatment with either catabolic enzyme inhibitors (BEC or 1MDT) or with blocking antibodies directed against TGF-β or PD-1 led to a statistically significant decrease in total urogenital tract (UGT) weight (FIG. 7A) and prostate weight (FIG. 7B). When taken in combination with the data set forth in Examples 11-13, these data suggest that following blockade of suppressive mediators, the TcR-I T cells initially infiltrate the prostate with effector functions capable of slowing tumor growth, but eventually become tolerized and lose this ability, resulting in restoration of tumor growth.

### Example 15

This example demonstrates that silencing expression of Foxo3a restores the capacity of TADCs to stimulate anti-prostate tumor responses and reduces their tolerogenicity.

RNA was isolated from DCs purified from TRAMP and WT mouse prostate tissue and gene expression was analyzed using Partek (St. Louis, Mo.) and Gene Portal software and verified by real-time PCR. TRAMP DCs express a 6-fold increase in foxo3a mRNA levels as compared to WT prostate DC. Increased FOXO3A protein levels were also detected in TADC compared to WT prostate DC by flow cytometric analysis.

A combination of siRNAs (SEQ ID NOs: 1-4) was used to silence expression. Protein lysates were assayed by Western blot to confirm gene silencing and showed that FOXO3A protein levels were slightly reduced at 24 hrs but were reduced to the low levels detected in WT prostate DC 48 hrs after siRNA transduction.

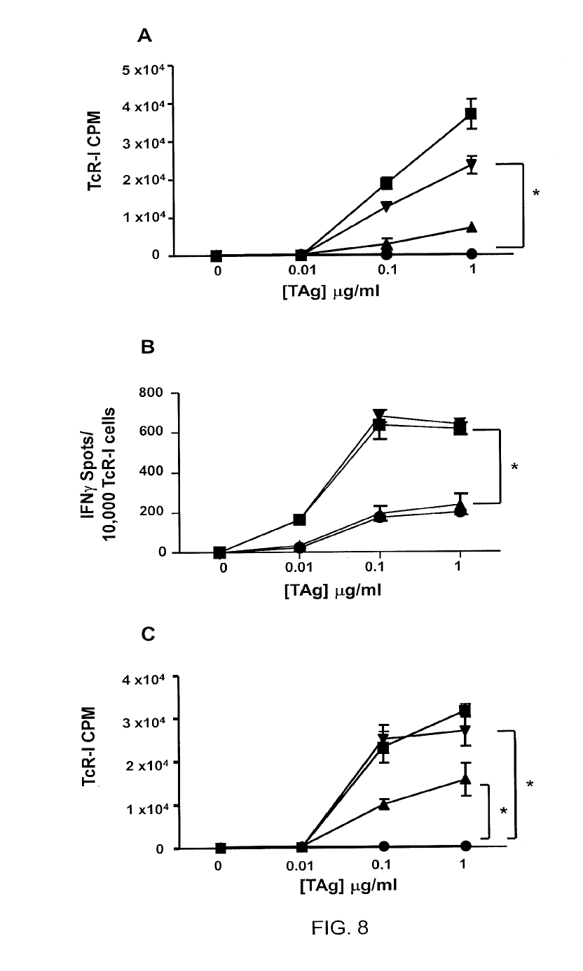

TADCs treated with foxo3a siRNA (SEQ ID NOs: 1-4) were used to stimulate naïve TcR-I T cells. TRAMP DCs were added to naïve TcR-I cells and proliferation was tested after 60 hours (FIG. 8A). Tolerization of T cells was tested 4 days after stimulation with TADC as measured by IFN-γ secretion (FIG. 8B), proliferation (FIG. 8C), and suppressor activity (Table 12).

TABLE 12

| | % Suppression (for each source of primary stimulation) | | | |
|---|---|---|---|---|
| Suppressor: | Untreated | TRAMP DC + | TRAMP DC + | |
| Responder | TRAMP DC | control siRNA | Foxo3a siRNA | WT DC |
| 2:1 | 97 | 99* | 50* | 10 |
| 1:1 | 90 | 80* | 40* | <5 |
| 1:2 | 15 | 60* | 20* | <5 |
| 1:4 | 15* | 10* | 10* | <5 |

Data are representative of 3 independent experiments (3 mice per group), mean ± s.d.,
*p < 0.01 (Student's t-test).

In contrast to TADCs treated with control siRNAs, that are poorly stimulatory, the foxo3a siRNA-treated TADCs were capable of inducing both strong proliferative (FIG. 8A) and cytokine (FIG. 8B) responses by TcR-I T cells. Moreover, foxo3a-silenced TADCs did not tolerize or induce suppressor activity in TcR-I cells (FIG. 8C and Table 12).

This example demonstrated that silencing expression of foxo3a with siRNAs restores the capacity of the TADCs to stimulate more potent anti-tumor responses and reduces their tolerogenicity.

### Example 16

This example demonstrates that targeting foxo3a expression in TADCs down-regulates several aspects of DC function related to immune suppression.

To understand how silencing foxo3a resulted in greater immunostimulatory function of TADCs, changes in TADC phenotype and gene expression profile were examined. Silencing foxo3a using the siRNAs SEQ ID NOs: 1-4 enhanced CD80 expression but did not impact CD86 levels. Further analysis revealed that reducing foxo3a levels markedly decreased expression of ido and arg and even further increased expression of il-6, a pleiotropic cytokine associated with T cell survival and inflammation, as shown in Table 13.

TABLE 13

Fold Change TRAMP/WT

| | | TRAMP foxo3a siRNA (48 |
|---|---|---|
| Gene | TRAMP | hour co-culture) |
| ARG | 4* | −3* |
| IDO | 16* | −3* |
| IL-6 | 6* | 21* |
| TNF-α | 2 | 3 |

Data representative of 3 independent experiments using 3 mice for each group, mean ± s.d.,
*p < 0.01 for TRAMP vs. TRAMP foxo3a siRNA.

Additionally, production of TGF-β was completely abrogated upon silencing of foxo3a, as shown in Table 14.

TABLE 14

| | |
|---|---|
| ng/ml lipopolysaccharide | ng/ml TGF-β |

| (LPS) | Control siRNA | foxo3a siRNA |
|---|---|---|
| 0 | 1500 | not detectable (n.d.) |
| 10 | 2500 | n.d. |
| 100 | 4750 | n.d. |

This drastic reduction in TGF-β may explain the profound reduction of TADC-induced suppressor activity by CD8⁺ T cells (see Table 12).

This example demonstrated that silencing foxo3a expression in DCs increases CD80 and il-6 expression and decreases arg, ido, and TGF-β expression, all of which are consistent with enhancing anti-tumor immunity.

### Example 17

This example demonstrates that TADCs from TRAMP mice receiving an adoptive transfer of TcR-II T cells are unable to tolerize TcR-I cells in vitro, and that transfer of TcR-II T cells significantly reduces expression of ido, arg, and foxo3a in DCs from the TRAMP prostate.

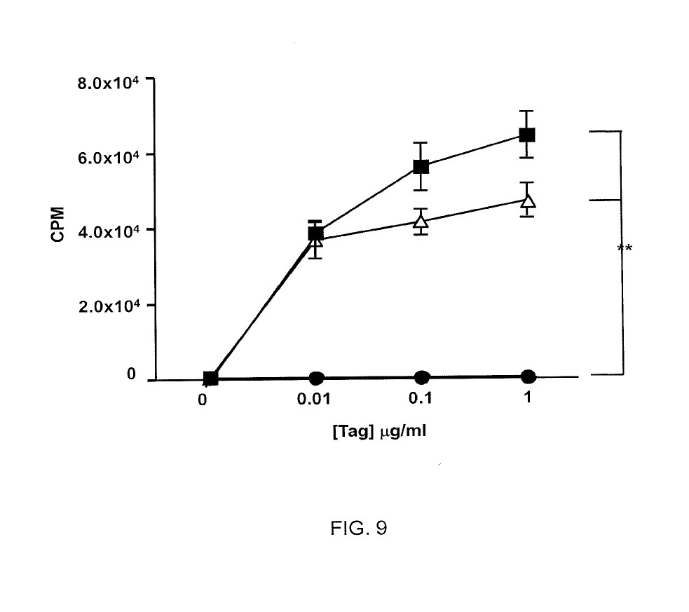

Naïve TcR-I T cells were cultured in vitro with prostatic DCs from WT or TRAMP mice that were administered antigen-specific CD4⁺ (TcR-II) T cells, and proliferative response (FIG. 9) and suppressive activity (Table 15) were tested.

TABLE 15

| Suppressor: | % Suppression (for each source of primary stimulation) | | |
|---|---|---|---|
| Responder | TRAMP DC | TRAMP + TcR-II | WT DC |
| 10:1 | 90 | 50 | 10 |
| 2:1 | 90 | 32 | 12 |
| 1:1 | 47 | 35 | 7 |
| 1:2 | 37 | 12 | 10 |

Data representative of 3 independent experiments with 3 mice per group, mean ± s.d.
*p < 0.01 (Student's t-test)

As shown in FIG. 9, TADCs isolated from TRAMP mice receiving an adoptive transfer of TcR-II cells were unable to tolerize TcR-I cells in vitro. Surprisingly, naïve TcR-I cells cultured with TADCs from TcR-II-transferred mice still maintained some suppressor functions, albeit to a lesser degree than TcR-I T cells cultured with TADCs from unmanipulated TRAMP mice (Table 15).

Expression of the genes ido, arg, and foxo3a in TADCs from TRAMP mice and TcR-II-transferred TRAMP mice was also measured (Table 16A).

TABLE 16A

Fold-change expression TRAMP/WT

| Gene | TRAMP | TRAMP + TcR-II |
|---|---|---|
| ido | 10* | 3* |
| arg | 24** | 1** |
| foxo3a | 11* | 5* |

Data are representative of 3 independent experiments using 3 mice for each group, mean ± s.d.
*p < 0.01,
**p < 0.0001 (TRAMP vs. TRAMP + TcR-II) (Student's t-test).

As shown in Table 16A, transfer of TcR-II T cells significantly reduced expression of ido, arg and foxo3a in DCs from the TRAMP prostate. However, foxo3a levels were not reduced to the level observed in WT prostate DCs, which might explain the residual ability to induce suppression by TcR-I cells.

To confirm that TcR-II cells directly act on TADC to alter their function, TADC cells were cultured with TcR-II cells and subsequently tested for tolerogenicity and gene expression. TcR-II cells were cultured with TADC for 24 hours with the indicated antigen dose prior to testing TADC tolerogenicity (Table 16B) or measuring gene expression of foxo3a, ido, and arg (Table 16C).

TABLE 16B

| | Proliferation (CPM) | | |
|---|---|---|---|
| | | TADC + TcR-II (0 | TADC + TcR-II (1 |
| [TAg] μg/ml | TADC | μg antigen) | μg antigen) |
| 0 | 0 | 0 | 0 |
| 0.01 | 0 | 1.0 × 10⁴ | 2.5 × 10⁴ |
| 0.1 | 0 | 1.2 × 10⁴ | 3.5 × 10⁴ |
| 1.0 | <0.5 × 10⁴ | 1.5 × 10⁴ | 3.6 × 10⁴ |

Data representative of 2 individual experiments.
p < 0.0001

TABLE 16C

Fold-change expression TRAMPAVT

| Gene | TRAMP | TRAMP + TcR-II |
|---|---|---|
| ido | 13 | <3* |
| arg | 23 | 13* |
| foxo3a | 8 | <3* |

Data are representative of 2 individual experiments,
*p < 0.001,
**p < 0.0001 (TRAMP vs. TRAMP + TcR-II) (Student's t-test).

Co-culture with TcR-II cells diminished the ability of TADC to induce tolerance in CD8+ T cells (Table 16B). Furthermore, TcR-II-stimulated DC had a significant reduction in expression of foxo3a, ido, and arg (Table 16C).

This example demonstrated that TADCs can be targeted in vivo to support enhanced T cell activation and effector functions and inhibition of foxo3a expression may significantly influence tolerogenicity of TADCs.

### Example 18

This example demonstrates that expression of the immuno-suppressive genes, fasl, ido, pd-11, stat3, and foxo3a, is up-regulated in TADCs from human tumor biopsies compared to DC from non-tumor prostatic tissues.

Gene expression and function was analyzed in human TADCs. Microarray analyses were performed, comparing the profiles of CD 123⁺ pDCs isolated from tumor and non-tumor specimens (Table 17).

TABLE 17

| | Fold-Change |
|---|---|
| Gene | Tumor/Non-Tumor |
| fasl | 5.2 |
| ido | 7.3 |
| pd-l1 | 3.1 |
| stat3 | 5.1 |
| foxo3a | 6.9 |

Fold-change values have corresponding p < 0.00001 (ANOVA). Data are representative of 5 independent microarrays for tumor and non-tumor biopsies.

The data demonstrate a profile consistent with that observed with TRAMP (mouse) TADC. Namely, a significant up-regulation of immuno-suppressive genes was observed, including fasl, ido, pd-11, stat3 and foxo3a in TADCs harvested from human prostate tumor biopsies compared to pDC from non-tumor prostatic tissues, as shown in Table 17. RT-qPCR confirmed human TADCs expressed approximately 8-fold higher levels of ido and 40-fold higher levels of foxo3a mRNA compared to pDCs isolated from non-tumor prostate biopsies, as shown in Table 18.

TABLE 18

| | Fold-Change Expression |
|---|---|
| Gene | Tumor/Non-Tumor |
| foxo3a | 43 |
| ido | 8 |
| arg 1 | 5 |

Flow cytometric analysis confirmed elevated expression of PD-L1 on human prostate TADCs.

This example demonstrated that fall, ido, pd-11, stat3, and foxo3a expression is up-regulated in TADCs from human prostate tumor biopsies compared to DC from non-tumor prostatic tissues.

### Example 19

This example demonstrates that silencing foxo3a enhances the ability of TADC to stimulate a proliferative response by T cells and abrogates the ability of human TADC to tolerize T cells and induce suppressive activity.

To determine whether foxo3a expression is required for tolerogenicity of human TADC, foxo3a expression was silenced using a combination of siRNAs (SEQ ID NOs: 1-4). siRNA-treated TADCs isolated from human prostate tumor tissues were used to stimulate (Table 19), tolerize (Table 20), or induce suppressor activity (Table 21) in autologous PBMCs in vitro using the CEF (CMV, EBV and Flu) viral peptide pool. Proliferation of primary (Table 19) or secondary (Table 20) responses was assessed using thymidine incorporation.

TABLE 19

| | CPM - Patient PBMC | | |
|---|---|---|---|
| | | | Tumor PDc + |
| | | Tumor pDC + | Negative Control |
| [CEF] μg/ml | Tumor pDC | foxo3a siRNA | siRNA |
| 0 | <500 | 1.0 × 10³ | <500 |
| 1 | <500 | 2.5 × 10³** | 500 |
| 2 | 750 | 4.5 × 10³** | 1.5 × 10³ |
| 5 | 1.0 × 10³ | 6.5 × 10³** | 2.0 × 10³ |

Data representative of 3 patient samples, mean ± s.d.
*p < 0.01,
**p < 0.001 (Student's t-test)

TABLE 20

| | CPM - Patient PBMC (for each source of primary stimulation) | | |
|---|---|---|---|
| [CEF] | TADC + negative | TADC + foxo3a | |
| μg/ml | control siRNA | siRNA | PBMC |
| 1 | 5.0 × 10³ | 1.0 × 10⁴ | 2.0 × 10⁴ |
| 2 | 5.0 × 10³* | 2.5 × 10⁴* | 4.5 × 10⁴ |
| 5 | 5.0 × 10³** | 5.0 × 10⁴** | 8.0 × 10⁴ |
| 10 | 5.0 × 10³** | 7.5 × 10⁴** | 1.2 × 10⁵ |

Data representative of 3 patient samples, mean ± s.d.
*p < 0.01,
**p < 0.001 (Student's t-test)

TABLE 21

| | % Suppression (for each source of primary stimulation) | | |
|---|---|---|---|
| Suppressor: | TADC + foxo3a | TADC + negative | |
| Responder | siRNA | control siRNA | PBMC |
| 2:1 | 25** | 55** | 5 |
| 1:1 | 25* | 45* | 10 |
| 1:2 | 18 | 30 | 10 |
| 1:4 | 12 | 15 | 11 |

Data representative of 2 patient samples, mean ± s.d.
*p < 0.01,
**p < 0.001 (TADC + foxo3a siRNA vs. TADC + negative control siRNA) (Student's t-test)

As seen in Table 19, silencing foxo3a (>greater than 90% reduction in gene expression) significantly enhanced TADC stimulation of T cells as indicated by an increase in proliferative response relative to priming by TADC treated with control siRNAs. Moreover, silencing foxo3a expression also abrogated the ability of human TADC to tolerize T cells and induce suppressive activity (Tables 20 and 21). Taken together, these data demonstrate that similar to TRAMP TADC, expression of foxo3a is critical for the immunosuppressive activities of TADC infiltrating human prostate cancer tissues.

This example demonstrated that silencing foxo3a gene expression using siRNAs enhanced the ability of TADC to induce T cell proliferation and abrogated the ability of human TADC to tolerize T cells and induce suppressive activity.

### Example 20

This example demonstrates that silencing expression of foxo3a reduces the tolerogenicity of TADCs.

Human prostate TADC or PBMC were untreated or treated with a combination of siRNAs (sifoxo3a RNA; SEQ ID NOs: 44-47) or scrambled negative control siRNA (siRNA(−)) as indicated in Table 22. To determine the ability of the treated or untreated TADC or PBMC to stimulate T cells, the cells were cultured with autologous peripheral blood T cells and CEF. T cell proliferation was measured as counts per minute (CPM). The results are shown in Table 22.

TABLE 22

| | Counts per Minute (CPM)-Patient PBMC | | | | |
|---|---|---|---|---|---|
| [CEF] | | | TADC + | TADC + | PBMC + |
| μg/ml | TADC | PBMC | siRNA(−) | sifoxo3a | sifoxo3a |
| 0 | <0.5 × 10⁴ | <0.5 × 10⁴ | <0.5 × 10⁴ | 0.5 × 10⁴ | <0.5 × 10⁴ |
| 1 | <0.5 × 10⁴ | 1.5 × 10⁴ | <0.5 × 10⁴ | 1.9 × 10⁴ | 2.9 × 10⁴ |
| 2 | <0.5 × 10⁴ | 2.8 × 10⁴ | <0.5 × 10⁴ | 5 × 10⁴ | 3.3 × 10⁴ |
| 5 | <0.5 × 10⁴ | 2.6 × 10⁴ | <0.5 × 10⁴ | 6 × 10⁴ | 3.5 × 10⁴ |

As shown in Table 22, treatment with anti foxo3a siRNAs (SEQ ID NOs: 44-47) improved the stimulatory capacity of TADCs.

### Example 21

This example demonstrates that silencing expression of foxo3a reduces the tolerogenicity of melanoma tumor TADCs and stimulates anti-melanoma tumor responses.

To determine whether foxo3a regulation of DC tolerogenicity is unique to prostate tumors, foxo3a expression and function in pDC isolated from melanoma tumor models was also assessed. TADC were isolated via magnetic beads coupled to anti-CD317 and assessed for FOXO3A expression by flow cytometry or tested for their ability to induce tolerance in TcR-MeI T cells. CD317+/CD11c+ cells isolated from B16 melanoma tumors had elevated levels of FOXO3A comparable to TRAMP TADC. T cell tolerance in TcR-MeI T cells was also induced, as shown in Table 23. Silencing foxo3a using SEQ ID NOs: 1-4 prevented induction of T cell tolerance (Table 23).

TABLE 23

| | Proliferation (CPM) for Each Source of Primary Stimulation | | | |
|---|---|---|---|---|
| TRP-2 | | | B16 TADC + | B16 TADC + |
| (μm) | Splenic pDC | B16 TADC | sifoxo3a | siRNA(−) |
| 0 | 0 | 0 | 0 | 0 |
| 1 | 3.5 × 10⁴ | 1.5 × 10⁴ | 2.5 × 10⁴ | 1.0 × 10⁴ |
| 2 | 6.0 × 10⁴ | 2.0 × 10⁴ | 4.5 × 10⁴ | 2.0 × 10⁴ |
| 5 | 6.5 × 10⁴ | 2.5 × 10⁴ | 7.5 × 10⁴ | 2.5 × 10⁴ |

p < 0.01 tumor pDC v. spleen, or sifoxo3a v. siRNA(−) control.

This example demonstrated that silencing expression of foxo3a with siRNAs reduces the tolerogenicity of B16 melanoma tumor TADCs.

### Example 22

This example demonstrates that melanoma tumor TADCs from foxo3a^{-/-} mice do not induce T cell tolerance.

To assess the role of foxo3a in the generation of tolerogenic TADC in vivo, B16 tumors were injected subcutaneously into WT and foxo3a−/− mice. TADC were isolated from B16 tumors in WT or foxo3a^{-/-} mice via magnetic beads coupled to anti-CD317. Phenotypically, TADC from B16 tumors growing in foxo3a^{-/-} mice displayed elevated levels of CD80 and CD86 and lower levels of ido gene expression compared to TADC isolated from B16 tumors growing in WT mice. The fold change of ido gene expression (WT/Foxo3a^{-/-}) was about 11.

Four days after stimulation by B16 TADC, cell proliferation responses were tested using antigen pulsed splenocytes. The results are shown in Table 24.

TABLE 24

Proliferation (CPM) (for each
source of primary stimulation)

| [TRP-2] μM | WT | Foxo3a^{-/-} |
|---|---|---|
| 1 | 0 | 0 |
| 2 | 0.5 × 10⁴ | 2.6 × 10⁴* |
| 5 | 1.0 × 10⁴ | 6.5 × 10⁴** |
| 10 | 1.7 × 10⁴ | 4.5 × 10⁴** |

Data representative of 3 independent experiments using 5 mice for each group, mean ± s.d.
*p < 0.01,
**p < 0.0001 (Student's t-test).

As shown in Table 24, TADC isolated from the foxo3a−/− mice did not tolerize TcR-MeI T cells.

This example demonstrated that in the absence of foxo3a, B16 melanoma tumor TADCs do not induce the tolerance of T cells.

### Example 23

This example demonstrates that deficiency of expression of foxo3a reduces the tolerogenicity of renal cancer (RENCA) TADCs.

FOXO3A expression and function in DC isolated from RENCA tumors was also assessed. TADC were isolated via magnetic beads coupled to anti-CD317 and assessed for FOXO3A expression by flow cytometry or tested for their ability to induce tolerance in TRP-2-specific T cells. CD317+/CD11c+ cells isolated from orthotopic RENCA tumors had elevated levels of FOXO3A comparable to TRAMP TADC. T cell tolerance in the T cells was also induced, as shown in Table 25. Silencing foxo3a using SEQ ID NOs: 1-4 prevented induction of T cell tolerance (Table 25).

TABLE 25

| | Proliferation (CPM) for Each Source of Primary Stimulation | | | |
|---|---|---|---|---|
| TRP-2 | | | RENCA | RENCA |
| Antigen | Normal | RENCA | TADC + | TADC + |
| (μm) | Kidney DC | TADC | siFoxo3a | siRNA(−) |
| 0 | 0 | 0 | 0 | 0 |
| 1 | 0.55 × 10³ | <0.1 × 10³ | 0.15 × 10³ | <0.1 × 10³ |
| 2 | 0.75 × 10³ | <0.1 × 10³ | 0.80 × 10³ | <0.1 × 10³ |
| 5 | 1.00 × 10³ | <0.1 × 10³ | 0.95 × 10³ | <0.1 × 10³ |

This example demonstrated that silencing expression of foxo3a with siRNAs reduces the tolerogenicity of renal tumor TADCs.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of enhancing an immune response to a cancer antigen in a mammal, which method comprises administering a cancer antigen to a mammal and inhibiting the activity of the foxo3a gene or gene product in dendritic cells in the mammal by administering an siRNA selected from the group consisting of (a) SEQ ID NOs: 44-47 and 1-4 and (b) siRNAs having at least 95% identity to any one of SEQ ID NOs: 44-47 and 1-4 and a nucleotide length of about 18 to about 30 to the mammal, thereby enhancing an immune response to the cancer antigen in the mammal.

2. A method of enhancing an immune response to a cancer antigen in a mammal, which method comprises:
(a) obtaining dendritic cells from the mammal;
(b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells;
(c) inhibiting the activity of the foxo3a gene or gene product in the dendritic cells by delivering an siRNA selected from the group consisting of (i) SEQ ID NOs: 44-47 and 1-4 and (ii) siRNAs having at least 95% identity to any one of SEQ ID NOs: 44-47 and 1-4 and a nucleotide length of about 18 to about 30 to the dendritic cells to produce dendritic cells having decreased foxo3a gene or gene product activity; and
d) administering the antigen-expressing dendritic cells having decreased foxo3a gene or gene product activity to the mammal, thereby enhancing an immune response to the cancer antigen in the mammal.

3. A method of enhancing an immune response to a cancer antigen in a mammal, which method comprises:
(a) obtaining T cells and dendritic cells from the mammal;
(b) causing the dendritic cells to express the cancer antigen by either (i) exposing the dendritic cells to the cancer antigen in culture under conditions promoting uptake and processing of the antigen, or (ii) transducing the dendritic cells with a nucleic acid sequence encoding the cancer antigen to produce antigen-expressing dendritic cells;
(c) inhibiting the activity of the foxo3a gene or gene product in the dendritic cells by delivering an siRNA selected from the group consisting of (i) SEQ ID NOs: 44-47 and 1-4 and (ii) siRNAs having at least 95% identity to any one of SEQ ID NOs: 44-47 and 1-4 and a nucleotide length of about 18 to about 30 to the dendritic cells to produce dendritic cells having decreased foxo3a gene or gene product activity;
(d) exposing the antigen-expressing dendritic cells having decreased foxo3a gene or gene product activity to the T cells; and
(e) administering the T cells to the mammal, thereby enhancing an immune response to the cancer antigen in the mammal.

4. The method of claim 2, wherein causing the dendritic cells to express the cancer antigen further comprises exposing the dendritic cells to granulocyte macrophage-colony stimulating factor (GM-CSF).

5. The method of Claim 1, wherein the activity of the foxo3a gene is inhibited by decreasing endogenous expression of the foxo3a gene.

6. The method of claim 1, wherein the immune response to the cancer antigen enhanced in the mammal is greater than the immune response to the cancer antigen in the absence of the administration of a foxo3a siRNA, dendritic cells, or T cells to the mammal.

7. The method of any claim 1, wherein enhancing an immune response to the cancer antigen comprises stimulating T cells in the mammal.

8. The method of claim 7, wherein stimulating T cells in the mammal comprises increasing T cell proliferation.

9. The method of claim 7, wherein stimulating T cells in the mammal comprises increasing interferon-gamma (IFN-γ) secretion by the T cells.

10. The method of claim 1, wherein administering the siRNA to the mammal increases dendritic cell CD80 expression.

11. The method of claim 1, wherein administering the siRNA to the mammal increases dendritic cell interleukin (IL)-6 expression.

12. The method of claim 1, wherein administering the siRNA to the mammal decreases dendritic cell arginase and/or transforming growth factor (TGF)-β expression.

13. The method of claim 1, wherein administering the siRNA to the mammal decreases dendritic cell indolamine-2-3-deoxygenase (IDO) expression.

14. The method of claim 1, wherein the cancer antigen is a prostate cancer antigen, a melanoma antigen, or a renal cancer antigen.

15. The method of claim 14, wherein the cancer antigen is a prostate cancer antigen selected from the group consisting of prostate-specific antigen (PSA), prostate-specific membrane antigen (PSMA), prostate stem cell antigen (PSCA), prostatic acid phosphatase (PAP), telomerase reverse transcriptase (TERT), survivin (BIRC5), and mucin-1 (MUC1).

16. The method of claim 15, wherein the cancer antigen is prostatic acid phosphatase (PAP).

17. The method of claim 15, wherein the cancer antigen is a melanoma antigen selected from the group consisting of gp100, MART-1, p15, mutant cyclin-dependent kinase 4 (CDK4), NY-ESO-1, MAGE 1, MAGE 2, MAGE 3, mesothelin, tyrosinase tumor antigen, tyrosinase related protein (TRP)-1, and TRP-2.

18. The method of claim 1, wherein the dendritic cells are tumor-associated dendritic cells (TADCs).

19. The method of claim 1, wherein administering the siRNA, dendritic cells, or T cells to the mammal comprises administering the siRNA, dendritic cells, or T cells directly into a tumor.

20. A method of suppressing an immune response to an autoimmune disease antigen in a mammal, which method comprises administering the autoimmune disease antigen to the mammal and increasing the activity of the foxo3a gene or gene product in dendritic cells in the mammal by administering a nucleic acid encoding foxo3a selected from the group consisting of (i) SEQ ID NOs: 5-6 and (ii) sequences having at least 95% identity to SEQ ID NO: 5 or 6 to the mammal, thereby suppressing an immune response to the autoimmune disease antigen in the mammal.

21. A method of suppressing an immune response to an autoimmune disease antigen in a mammal, which method comprises
(a) obtaining dendritic cells from the mammal;
(b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells;
(c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity; and
(d) administering the antigen-expressing dendritic cells having increased foxo3a gene or gene product activity to the mammal, thereby suppressing an immune response to the autoimmune disease antigen in the mammal.

22. A method of suppressing an immune response to an autoimmune disease antigen in a mammal, which method comprises
(a) obtaining T cells and dendritic cells from the mammal;
(b) causing the dendritic cells to express the autoimmune disease antigen by either (i) exposing the dendritic cells to the autoimmune disease antigen in culture under conditions promoting uptake and processing of the antigen, or (ii) transducing the dendritic cells with a nucleic acid sequence encoding the autoimmune disease antigen to produce antigen-expressing dendritic cells;
(c) increasing the activity of the foxo3a gene or gene product in the dendritic cells by delivering a nucleic acid encoding foxo3a selected from the group consisting of (i) SEQ ID NOs: 5-6 and (ii) sequences having at least 95% identity to SEQ ID NO: 5 or 6 to the dendritic cells to produce dendritic cells having increased foxo3a gene or gene product activity;
(d) exposing the antigen-expressing dendritic cells having increased foxo3a gene or gene product activity to the T cells; and
(e) administering the T cells to the mammal, thereby suppressing an immune response to the autoimmune disease antigen in the mammal.

23. The method of claim 20, wherein the activity of the foxo3a gene is increased by increasing endogenous expression of the foxo3a gene.

24. The method of claim 20, wherein the immune response to the autoimmune disease antigen suppressed in the mammal is less than the immune response to the autoimmune disease antigen in the absence of the administration of a nucleic acid encoding foxo3a to the mammal.

25. The method of claim 20, wherein suppressing an immune response to the autoimmune disease antigen comprises suppressing T cell activity in the mammal.

26. The method of claim 25, wherein suppressing T cell activity in the mammal comprises decreasing T cell proliferation.

27. The method of claim 25, wherein suppressing T cell activity in the mammal comprises decreasing interferon-gamma (IFN-γ) secretion by the T cells.

28. The method of claim 20, wherein administering the nucleic acid encoding foxo3a to the mammal decreases dendritic cell CD80 expression.

29. The method of claim 20, wherein administering the nucleic acid encoding foxo3a to the mammal decreases dendritic cell interleukin (IL)-6 expression.

30. The method of claim 20, wherein administering the nucleic acid encoding foxo3a to the mammal increases dendritic cell arginase expression.

31. The method of claim 20, wherein administering the nucleic acid encoding foxo3a to the mammal increases dendritic cell indolamine-2-3-deoxygenase (IDO) expression.

32. The method of claim 20, wherein the autoimmune disease antigen is selected from the group consisting of myelin basic protein (MBP), myelin proteolipid protein (PLP1), myelin oligodendrocyte glycoprotein (MOG), insulin (INS), glutamic acid decarboxylase (GAD1), and beta-cell zinc transporter ZvT8 (SLC30A8).

33. A method of treating or preventing cancer, which method comprises enhancing an immune response to a cancer antigen in a mammal according to the method of claim 1.

34. A method of treating or preventing an autoimmune disease, which method comprises suppressing an immune response to an autoimmune disease antigen in a mammal according to the method of claim 20.
